(19) United States
(12) Patent Application Publication
(10) Pub. No.: US 2021/0268203 A1
(43) Pub. Date: Sep. 2, 2021

(54) MEDICAL DEVICE CONNECTORS

(71) Applicant: ConceptoMed AS, Ballstad (NO)

(72) Inventors: Christian Mide, Ballstad (NO); Jimmy Gidö Schön, Ballstad (NO)

(21) Appl. No.: 17/265,739

(22) PCT Filed: Aug. 5, 2019
(86) PCT No.: PCT/EP2019/071056

§ 371 Date: Feb. 3, 2021

(30) Foreign Application Priority Data
Aug. 3, 2018 (GB) 1812698.7
Oct. 8, 2018 (GB) 1816395.6

(51) Int. Cl. A61M 5/34

(52) U.S. Cl. CPC A61M 5/344; A61M 5/347; A61M 5/346

(57) ABSTRACT

A medical device connector includes a fluid transfer tip including a tapered surface for creating a friction fitting between the fluid transfer tip and a hub. A separable collar includes a first and second arcuate segments. The second segment includes an engagement feature for engaging with a corresponding engagement feature of a hub. A moveable disconnection member is arranged such that movement of the disconnection member moves the second segment of the separable collar from a first closed collar configuration position, to a second position, corresponding to an open collar configuration, in which the second segment is moved relative to the fluid transfer tip and the first segment, so as to allow disengagement of the engagement feature from the corresponding engagement feature. Movement of the disconnection member also releases the friction fitting between the fluid transfer tip and the hub.

This application is entitled to the benefit of, and incorporates by reference essential subject matter disclosed in PCT Application No. PCT/EP2019/071056 filed on Aug. 5, 2019, which claims priority to GB Patent Appln. No. 1812698.7 filed Aug. 3, 2018 and GB Patent Appln. No. 1816395.6 filed Oct. 8, 2018, which are hereby incorporated by reference in their entireties.

### BACKGROUND OF THE INVENTION

### 1. Technical Field

The present invention relates to medical device connectors and related systems.

### 2. Background Information

The Applicant has previously devised solutions for easily disconnecting a contaminated needle from a syringe (or other fluid transfer device) using one hand as disclosed in WO 2013/164358, WO 2014/020090, WO 2015/014914 and WO 2016/162571. The Applicant's “LuerJack” system typically uses a pivoting disconnecting member, e.g. lever member, to separate the needle hub from the syringe. By utilizing a lever member the practitioner can, in a one-handed operation, more easily disconnect the needle hub from the syringe and reduce the risk of needlestick injuries. However, the “LuerJack” system has been designed for standard Luer connections and it is now appreciated that Luer-type connections do not represent the full range of clinical procedures that may benefit from one-handed operation.

Conventional needle hubs are usually standard Luer parts and many fluid transfer devices have been designed in the past to include a Luer-type connector. More By utilizing a lever member the practitioner can, in a one-handed operation, more easily disconnect recently, however, the ISO 80369 series of small-bore connector standards for fluid connectors in healthcare applications has been expanded to define a number of distinct connector types for different clinical applications. In the present ISO 80369 series: a Luer connector is compliant with ISO 80369-7 for connections in intravascular applications or hypodermic connections in hypodermic applications of medical devices and accessories; an ENFit connector is compliant with ISO 80369-3 for connections on enteral medical devices and accessories; and an NRFit connector is compliant with ISO 80369-6 for connections in neuraxial applications. The aim of this series of standards is to prevent misconnections between fluid transfer lines for different clinical uses, e.g. between enteral feeding tubes and IV lines. This series of standards may be further expanded in future.

These different standard connectors generally require a two-handed operation to achieve connection or disconnection e.g. by screwing together or unscrewing the male and female parts. There is no convenient, one-handed way of disconnecting a standard medical device connector which is also designed to prevent misconnections between different connector types.

The present invention aims to address or at least mitigate one or more of the problems outlined above.

### SUMMARY OF THE INVENTION

When viewed from a first aspect, the present invention provides a medical device connector for connecting, in use, to a hub, the medical device connector comprising: a fluid transfer tip comprising a tapered surface for creating a friction fitting between the fluid transfer tip and a hub connected to the fluid transfer tip in use; a separable collar, comprising at least a first arcuate segment and a second arcuate segment, which extend at least partially around the fluid transfer tip, wherein the first segment is arranged in a fixed position relative to the fluid transfer tip and the second segment is moveably mounted relative to the fluid transfer tip and the first segment; wherein the second segment comprises an engagement feature for engaging with a corresponding engagement feature of a hub connected to the fluid transfer tip in use; the separable collar having a closed configuration in which the second segment is positioned relative to the fluid transfer tip so as to extend around a hub connected to the tip in use and to engage with the hub by engagement between the engagement feature of the second segment and the corresponding engagement feature of the hub, and an open configuration in which the second segment is moved relative to the fluid transfer tip and the first segment, so as to allow disengagement of the engagement feature from the corresponding engagement feature; and a moveable disconnection member arranged such that movement of the disconnection member moves the second segment of the separable collar from a first position, corresponding to the closed configuration of the separable collar, to a second position, corresponding to the open configuration of the separable collar, and wherein movement of the disconnection member also releases the friction fitting between the fluid transfer tip and the hub by advancing the hub along the tapered surface of the fluid transfer tip.

It will be appreciated that such a medical device connector can be made compatible with any one of the ISO 80369 series of small-bore connector standards by appropriate choice of dimensions for the fluid transfer tip and separable collar. In particular, as is described below, the medical device connector is preferably compatible with one of the standards selected from ISO 80369-3, ISO 80369-6 or ISO 80369-7. A user will only be able to connect those hubs that meet the same standard and hence the device inherently prevents misconnections. In addition, the device provides the advantages of one-handed operation by enabling a user to operate the disconnection member in a single action that moves the separable collar into the open configuration to disengage the hub and releases the friction fitting between the fluid transfer tip and the hub.

The device is made relatively simple by having the first arcuate segment arranged in a fixed position relative to the fluid transfer tip. The disconnection member then only has to operate to move the second arcuate segment relative to the fluid transfer tip and the first segment. Furthermore, the Applicant has recognized that it is advantageous for the first segment to be fixed as this maintains a fixed dimensional relationship with the fluid transfer tip even when the separable collar is in the open configuration. Thus, even in the open configuration, a user is generally prevented from misconnecting the wrong type of hub to the fluid transfer tip. This can be contrasted with a collar that completely separates in all directions, leaving an exposed fluid transfer tip to which a user might be able to misconnect a hub even though the collar would likely not be able to move properly back to its closed configuration. This is important for avoiding user confusion.

A benefit of the separable collar comprising at least first and second arcuate segments, rather than more localized engagement features, is that the collar has a certain angular extent. Firstly, this means that the collar (at least in its closed configuration) looks the same as, or similar to, the collar of a standard connector despite the fact that the collar is separable. Secondly, the arcuate extent of the first and second segments can contribute to the stiffness of the collar. This can be important for ensuring that a user is not able to forcibly misconnect the wrong type of hub. The arcuate extent of the second segment can also make the engagement feature(s) more robust. This will be described in more detail below.

It will be appreciated that, in at least some embodiments, the first and second arcuate segments extending at least partially around the fluid transfer tip means that the first and second arcuate segments are arranged concentrically relative to the fluid transfer tip. The first arcuate segment may be arranged at a first radial distance from the fluid transfer tip and the second arcuate segment may be arranged at a second radial distance from the fluid transfer tip. In at least some embodiments, the first and second radial distances may be substantially the same. The collar may therefore appear symmetrically arranged about the fluid transfer tip.

It will also be appreciated that, while the first and second arcuate segments are generally arcuate in shape, it is not necessary for the first and second arcuate segments to have an outer profile that is constantly curved. In some embodiments, the first and/or second arcuate segments may have an outer profile that is curved, for example an outer profile at a fixed radius from the fluid transfer tip. In some embodiments, the first and/or second arcuate segments may have an outer profile that is partly curved and partly straight. For example, at least one of the first and second arcuate segments may have an outer profile that is generally U-shaped or J-shaped. In one set of embodiments, the second arcuate segment is U-shaped and the first arcuate segment sits inside the U-shaped second segment in the closed configuration. In such embodiments, the first and second arcuate segments may not mate together in the closed configuration. The second arcuate segment may have a much larger angular extent than the first arcuate segment, which can help to ensure the second arcuate segment is stiff and holds its shape when moved between the closed and open configurations of the collar.

In addition to the arcuate extent of the first and second segments, the arrangement of the first and second segments around the fluid transfer tip can help to prevent misconnections. In at least some embodiments, the fluid transfer tip extends along an axis that is surrounded by the separable collar, for example the separable collar may be coaxial with the fluid transfer tip, and the fluid transfer tip extends no further than an outer surface of the separable collar. This means that a compatible hub must be dimensioned to fit inside the collar before it can connect onto the fluid transfer tip. If the hub is not suitably dimensioned, then it will come into contact with an outer surface of the collar and be prevented from reaching the fluid transfer tip.

In at least some embodiments, in addition or alternatively, the fluid transfer tip extends along an axis that is surrounded by the separable collar, for example the separable collar may be coaxial with the fluid transfer tip, and the separable collar extends substantially all the way around the fluid transfer tip in the closed configuration. In such embodiments, the first and second arcuate segments may mate together in the closed configuration so as to form a closed collar extending substantially all the way around the fluid transfer tip. In other words, the separable collar may appear as a complete 360° collar in the closed configuration. This helps to prevent user confusion and attempted misconnections, and can make the collar stiff in its closed configuration so that any attempt to misconnect the wrong type of hub is resisted by the 360° angular extent of the first and second segments.

In embodiments where the first and second arcuate segments mate together in the closed configuration, this may be achieved in a number of ways. For example, the first and second arcuate segments may be arranged to interlock. In at least some embodiments, one of the first arcuate segment or second arcuate segment comprises a tongue portion extending therefrom, and the other of the first arcuate segment or second arcuate segment comprises a receiving portion shaped to receive the tongue portion such that, in the closed configuration, the first arcuate segment and second arcuate segment form a closed collar extending substantially all the way around the fluid transfer tip. In at least some embodiments, at least one of the first and second arcuate segments comprises a latching feature or detent arranged to latch onto or hold the other of the first or second arcuate segments when in the closed configuration. As will be appreciated, this will also achieve an interlocking arrangement as described above. Such a latching feature or detent may assist in holding the first and second arcuate segments together in the closed configuration. This may further help to prevent misconnections by preventing a user from being able to forcibly separate the first and second arcuate segments when attempting to attach a non-compatible hub. The other of the first and second arcuate segments may comprise a cooperating feature onto which the latching feature or detent may engage. Such a latching feature or detent may comprise a protrusion extending from at least one of the first and second arcuate segments arranged to engage with a corresponding feature, for example in the form of a recess provided on the other first or second arcuate segment.

In some other embodiments, the separable collar may not extend substantially all the way around the fluid transfer tip in the closed configuration. In such embodiments, the first and second arcuate segments may be spaced from one another in the closed configuration so as to form a partially open collar extending around the fluid transfer tip. In at least some embodiments, in addition or alternatively, at least one of the first arcuate segment or second arcuate segment may comprise multiple portions that are spaced from one another along an arcuate extent of the segment. Even in the closed configuration, the separable collar may appear as a broken rather than continuous collar. However, it will be appreciated that the arrangement of the multiple portions around the fluid transfer tip can still act to prevent an incompatible hub from being misconnected onto the tip, as it is the overall spacing between the collar and the tip that determines whether a hub can be accommodated to form a connection.

The Applicant has recognized that it is most important for the first arcuate segment to have a set spacing from the fluid transfer tip as the first segment is arranged in a fixed position and this spacing always ensures that the wrong type of hub cannot be misconnected onto the tip. Thus, in a preferred set of embodiments, at least the first arcuate segment is arranged in a fixed position having a predefined spacing from the tapered surface of the fluid transfer tip. Furthermore, in at least some embodiments, the second arcuate segment is arranged at a predefined spacing from the tapered surface of the fluid transfer tip when the collar is in the closed configuration. The spacing between the first/second arcuate segment and the tapered surface may be predefined to accommodate a specific type of hub, for example predefined in accordance with one of the ISO 80369 series of small-bore connector standards mentioned above.

In some embodiments, the predefined spacing may be compliant with ISO 80369-7 for connections in intravascular applications or hypodermic connections in hypodermic applications of medical devices and accessories. The standard ISO 80369-7:2016 specifies dimensions and requirements for the design and functional performance of Luer connectors intended to be used for connections in intravascular applications or hypodermic connections in hypodermic applications of medical devices and accessories. These Luer connectors have a standard 6% tapered surface to provide a so-called Luer Slip connection, and optionally include a threaded collar to provide a standard Luer Lock connection. Although standard Luer Slip or Luer Lock connections use a male tapered tip that fits inside a female hub or adaptor, it is envisaged that this could be reversed and the fluid transfer tip could be a female part having an internal taper to create the friction fitting with a corresponding male hub.

In some other embodiments, the predefined spacing may be compliant with ISO 80369-3 for connections on enteral medical devices and accessories. Compatible enteral medical devices include enteral feeding sets, enteral drainage sets, enteral syringes, and patient interface devices including access ports. For example, ENFit connectors may comprise a male connector tip and a coaxial connection collar. The male connector tip may have a tapered lead-in portion. The coaxial connection collar may be internally threaded. Such ENFit connectors are dimensioned to prevent misconnections with Luer connectors. Some examples of such ENFit connectors are disclosed in US 2016/0279032 and US 2017/0014616, the contents of which are hereby incorporated by reference.

In some other embodiments, the predefined spacing may be compliant with ISO 80369-6 for connections in neuraxial applications. Neuraxial applications involve the use of medical devices intended to administer medications to neuraxial sites, wound infiltration anesthesia delivery, and other regional anesthesia procedures or to monitor or remove cerebro-spinal fluid for therapeutic or diagnostic purposes. For example, NRFit connectors may comprise a male tapered tip surrounded by a coaxial collar that is internally threaded. Such NRFit connectors are dimensioned to prevent misconnections with Luer connectors.

In addition to the arrangement of the separable collar around the fluid transfer tip, the one or more engagement features provided by the second segment can be designed for compatibility with a specific type of hub. In some embodiments, it may be desirable for the engagement feature(s) to engage with the different corresponding engagement features of multiple hubs, even hubs compliant with different ones of the ISO 80369 series of small-bore connector standards. This can reduce the variation between devices intended for use with different standard hubs. For example, the engagement feature(s) may comprise at least one latch member. A latch member may be able to generally engage with the different corresponding engagement features of multiple hubs. A latch member may be able to grip onto a corresponding engagement feature in the form of a flange or thread of a hub. The latch member may take the form of a tooth or hook.

However, as will be appreciated from the disclosure above, a Luer Lock hub, an ENFit hub or an NRFit hub made in compliance with one of the ISO 80369 series of small-bore connector standards is dimensioned to connect onto the fluid transfer tip while engaging with a coaxial collar that is threaded. Thus, for ease of connection and to avoid user confusion, the separable collar is preferably designed to provide a threaded engagement with a hub connected in use. A user may need two hands to screw the hub into threaded engagement with the medical device connector, but operation of the disconnection member can provide for convenient one-handed release of the hub.

Furthermore, the Applicant has realized that a threaded engagement feature may be able to positively engage with a flange of a hub as well as with a corresponding threaded engagement feature as found in ISO80369-compliant hubs. In one or more embodiments, the second segment comprises an engagement feature comprising at least one threaded portion for engaging with a corresponding threaded portion or flange of a hub connected to the fluid transfer tip in use. The at least one threaded portion may comprise a helical thread. The threaded portion may be continuous or discontinuous. Whether the threaded portion is continuous or discontinuous, the nature of a threaded portion means that a user may need to twist or screw a hub into engagement with the second segment when the separable collar is in its closed configuration.

In at least some embodiments, the threaded portion comprises one or more threads protruding from an internal surface of the second segment. As is conventional, the second segment may comprise a cylindrical internal surface and one or more threads may protrude from a portion of the cylindrical internal surface.

More generally, the second segment may comprise any engagement feature or set of engagement features in a helical arrangement extending around the fluid transfer tip. Such a helical arrangement may effectively function like a threaded portion by engaging with a corresponding threaded portion or flange of a hub connected to the fluid transfer tip in use. Thus, in one or more embodiments, the second segment comprises one or more engagement features in a helical arrangement extending around the fluid transfer tip. The helical arrangement may require a user to twist or screw a hub into engagement with the second segment when the separable collar is in its closed configuration. The helical arrangement may be continuous or discontinuous.

The threaded portion or helical arrangement may extend fully across an internal surface of the second segment, or the threaded portion or helical arrangement may only extend partially across an internal surface of the second segment. Thus, as compared to the threaded collar of a standard connector, the threaded portion or helical arrangement may have a reduced extent, which can assist with disengagement when the second segment is moved to its second position corresponding to the open configuration of the separable collar. In at least some embodiments, the engagement feature(s) are static relative to the second arcuate segment. For example, the engagement feature(s) may comprise at least one fixed thread. This can ensure a robust engagement, especially for threaded engagement.

In at least some other embodiments, the engagement feature(s) are displaceable at least relative to the second segment. For example, the engagement feature(s) may comprise at least one displaceable latch member. This can assist with a push-on type of engagement.

Regardless of the form of the engagement feature(s), e.g. a latch member or threaded portion, the second segment may define an internal surface extending around the fluid transfer tip, e.g. coaxial with the tip, and the engagement feature(s) may protrude or project from the internal surface towards the fluid transfer tip. In at least some embodiments, the engagement feature(s) may project radially from the internal surface.

As mentioned above, in at least some embodiments the engagement feature(s) may comprise at least one latch member. A latch member is any member protruding from an internal surface of the second segment to positively engage with (e.g. grip onto) a corresponding engagement feature of a hub in use. In a hybrid set of embodiments, the second segment comprises one or more latch members in a helical arrangement extending around the fluid transfer tip. Such arrangements combine a gripping function from the latch member(s) with a screw function from the helical arrangement. As before, the helical arrangement may require a user to twist or screw a hub into engagement with the second segment when the separable collar is in its closed configuration.

In at least some other embodiments, it may be desirable to avoid the need for a user to twist or screw a hub into engagement with the second segment. The open configuration of the separable collar may be exploited so that a user connects a hub to the fluid transfer tip while the second segment is moved away from the fluid transfer tip. When the second segment is moved to bring the collar into its closed configuration, the engagement feature(s) may then be brought into positive engagement with the corresponding engagement feature(s) of the hub. Without the engagement feature(s) being in a helical arrangement, it may still be beneficial for the engagement feature(s) to have an angular extent around the fluid transfer tip in order to provide secure engagement. In one or more embodiments, the engagement feature comprises one or more arcuate portions extending from a surface of the second segment. The arcuate portion(s) may be arranged concentrically around the fluid transfer tip.

As mentioned above, the engagement feature(s) may protrude to latch onto a corresponding engagement feature of a hub. In some embodiments such a latching engagement may be reversed, for example the corresponding engagement feature(s) of the hub may comprise a latch member and the second segment may comprise an engagement feature comprising a latch keeper. More generally, it will be appreciated that the engagement feature(s) may not protrude outwardly from an internal surface of the second segment but may instead be imprinted or impressed into an internal surface of the second segment. For example, the engagement feature(s) may comprise at least one groove.

In addition to providing for engagement with a hub connected to the fluid transfer tip in use, the Applicant has recognized that the engagement feature(s) of the second segment may also be arranged to provide a deceleration effect when the separable collar is moving between its closed and open configurations upon operation of the disconnection member. The dual function of the disconnection member is not only to open the separable collar but also to advance the hub along the tapered surface of the fluid transfer tip to thereby release the friction fitting. As the disconnection member is pushing the hub along the fluid transfer tip, the hub may be forcibly released and there is a risk that the hub shoots away from the device in an uncontrolled manner. This can be mitigated by the separable collar at least partially resisting advancement of the hub along the fluid transfer tip. Thus, in a preferred set of embodiments, the separable collar comprises a deceleration feature. In some embodiments, this deceleration feature may be provided by the engagement feature(s) of the second segment. For example, a threaded portion may be arranged on an internal surface of the second segment so as to at least partially resist advancement of the hub at the same time as disengaging from the corresponding threaded portion of the hub.

As discussed above, the primary purpose of the first arcuate segment being in a fixed position is to define a fixed spacing from the fluid transfer tip that prevents misconnections. However, the Applicant has appreciated that it may be better for the first segment to provide a deceleration effect than the second segment, so that deceleration is decoupled from the disengagement that occurs when the separable collar is moving between its closed and open configurations upon operation of the disconnection member. Thus, in at least some embodiments, the first segment comprises a deceleration feature. This may be instead of, or in addition, to any deceleration feature provided by the engagement feature(s) of the second segment. The deceleration feature is any suitable feature arranged to interact with the hub so as to at least partially resist advancement of the hub along the fluid transfer tip.

In such embodiments, the deceleration feature may comprise at least one protrusion extending from the first segment in a direction towards the fluid transfer tip such that it interacts with a hub as it is advanced along the fluid transfer tip by the disconnecting member. The protrusion(s) may comprise a high friction material such as rubber or silicone. In some embodiments, the deceleration feature comprises multiple protrusions extending from the first segment. The multiple protrusions may be spaced apart along the fluid transfer tip, for example resulting in multiple deceleration stages as a hub is advanced along the tip.

In such embodiments, in addition or alternatively, the deceleration feature may be at least partially deformable such that as the hub is advanced across the deceleration feature, the deceleration feature deforms to allow the hub to advance along the fluid transfer tip. In such embodiments, in addition or alternatively, the deceleration feature may be at least partially displaceable such that as the hub interacts with the deceleration feature, the deceleration feature displaces to allow the hub to advance along the fluid transfer tip. The first segment, albeit in an overall fixed position relative to the fluid transfer tip, may comprise a displaceable portion that acts as the deceleration feature.

The Applicant has recognized that a collar providing a deceleration effect is advantageous in its own right, regardless of whether there is also provided a positive engagement between the collar and a hub connected to the fluid transfer tip in use. Thus, when viewed from a second aspect, the present invention provides a system comprising a hub and medical device connector; wherein the hub comprises: a connection feature arranged at a first end of the hub for connecting the hub to the medical device connector; and wherein the medical device connector comprises: a fluid transfer tip comprising a tapered surface for creating a friction fitting between the fluid transfer tip and the connection feature on the hub when the hub is connected to the medical device connector; a collar arranged to extend at least partially around the fluid transfer tip, and around at least part of the hub when the hub is connected to the fluid transfer tip, and wherein the collar comprises a deceleration feature; and a disconnection member arranged such that when the hub is connected to the fluid transfer tip, movement of the disconnection member from a first position to a second position advances the hub along the fluid transfer tip so as to release the friction fitting between the fluid transfer tip and the hub and wherein the advancement of the hub along the fluid transfer tip is at least partially resisted by an interaction between the deceleration feature and the hub.

It will be recognized that, in at least some embodiments, the collar may be a fixed collar rather than a separable collar and there may be no engagement between the collar and the hub. This means that the disconnection member, which may allow for one-handed operation, solely acts to release the friction fitting between the fluid transfer tip and the hub. However, the presence of the collar is still important as the predefined spacing between the collar and the tip can prevent user confusion and misconnections from occurring. An advantage of the absence of engagement features is that a standard Luer Slip hub can be connected onto the tip and take advantage of one-handed disconnection with deceleration.

In other embodiments, the collar is separable, comprising first and second arcuate segments with the features as described above. Thus, in at least some embodiments of this second aspect, the collar is a separable collar, comprising at least a first arcuate segment and a second arcuate segment, which extend at least partially around the fluid transfer tip. Preferably the first segment is arranged in a fixed position relative to the fluid transfer tip and the second segment is moveably mounted relative to the fluid transfer tip and the first segment. In some preferred embodiments: the second segment comprises an engagement feature for engaging with a corresponding engagement feature of a hub connected to the fluid transfer tip in use; the separable collar having a closed configuration in which the second segment is positioned relative to the fluid transfer tip so as to extend around a hub connected to the tip in use and to engage with the hub by engagement between the engagement feature of the second segment and the corresponding engagement feature of the hub, and an open configuration in which the second segment is moved relative to the fluid transfer tip and the first segment, so as to allow disengagement of the engagement feature from the corresponding engagement feature; and the moveable disconnection member arranged such that movement of the disconnection member moves the second segment of the separable collar from the first position, corresponding to the closed configuration of the separable collar, to the second position, corresponding to the open configuration of the separable collar.

There will now be described some features of the disconnection member that are generally applicable in embodiments of either of the first or second aspects of the invention.

In one or more embodiments, the disconnection member is resiliently biased into the first position. This can help to ensure that the default state of the medical device connector is one in which the disconnection member is not interfering with the friction fitting, and optionally the positive engagement, with a hub connected to the fluid transfer tip in use. A user must actively operate the disconnection member to overcome the resilient bias and cause the hub to be released.

The disconnection member may be resiliently biased by a spring member or by a natural resilient bias, for example resulting from the disconnection member being deformed from a relaxed state. In at least some embodiments, the disconnection member is moveably mounted such that movement of the disconnection member from the first position to the second position deforms the disconnection member so as to result in a resilient bias that tends to return the disconnection member to the first position. For example, the medical device connector may comprise a body member and the disconnection member may be moveably mounted to the body member with features operating between the disconnection member and the body member which interact to inhibit the disconnecting member from moving until a force is applied to the disconnecting member by a user to deform the disconnection member and overcome the interaction. This is described in more detail in the Applicant's published application WO 2016/162571, the contents of which are hereby incorporated by reference in their entirety.

In one or more embodiments, the disconnection member is locked in the first position until actively released. For example, the medical device connector may comprise a body member and the disconnection member may be prevented from moving out of the first position by a locking interaction between the disconnection member and the body member. In such embodiments, the device may further comprise a release member arranged to release the disconnection member from being locked in the first position. In addition, the release member may also operate to move the disconnection member out of the first position. Optionally, in some further embodiments, the release member is further arranged to drive movement of the disconnection member from the first position to the second position. This may be achieved, for example, through an interaction between lugs or side legs on the release member and side walls of the disconnection member. In a set of embodiments, the release member comprises deformable side legs which extend towards and rest on an outer surface of the body member and arranged such that, as the release member is pressed down towards the body member, the side legs are forced to splay outwards around the body member to contact the disconnection member and further operation of the release member thereby drives movement of the disconnection member.

The release member may be any manually operable member that operates to overcome the locking interaction between the disconnection member and the body member. The Applicant has recognized that it is desirable to prevent accidental operation of the release member, so that the disconnection member is locked in the first position until intentionally released by a user. The release member may comprise a resiliently-biased button, so that a user has to apply sufficient force to over the resilient bias before the button operates. This has the advantage of tactile feedback when a user rests a digit on the button and makes an active decision to apply a pressing force. Thus, in at least some embodiments, the medical device connector comprises a body member and the release member is mounted to the body member in a pre-stressed state. This may be achieved, for example, by the release member comprising deformable side legs which extend towards and rest on an outer surface of the body member and arranged such that, as the release member is initially pressed down towards the body member, the side legs are forced to splay outwards around the body member to put the release member in a pre-stressed state and the disconnection member comprising a latch arranged to hold the release member in the pre-stressed state.

In one or more embodiments, the disconnection member comprises a disconnection portion arranged to advance along the tapered surface of the tip to advance and push off the hub from the friction fitting. The disconnection portion may be forked, comprising a pair of fork legs extending either side of the fluid transfer tip. This can help to ensure a balanced disconnection force is applied to a hub connected to the tip in use. At least in embodiments wherein the first segment is fixed in position, the first segment may comprise two grooves for receipt of the fork legs. During movement of the disconnection member, the two grooves may receive the fork legs.

In some embodiments, the disconnection portion may be provided by a threaded portion. For example, as described above, the second segment may comprise at least one threaded portion for engaging with a corresponding threaded portion of a hub connected to the fluid transfer tip in use. When the second segment is moved between the first and second positions, the threaded portion may advance along the tapered surface of the fluid transfer tip to at least partially act as a disconnection portion. Even if the second segment is part of disconnection member in the form of a pivoting lever member, the threaded portion may act as a disconnection portion during pivotal movement to achieve the open configuration of the collar.

In one or more embodiments, the disconnection member is a pivotally mounted lever member, e.g. pivotally mounted to a body member of the medical device connector. The lever member may comprise a rearward portion that can be pivoted down towards the body member. In at least some embodiments, the rearward portion comprises a pair of side walls arranged to pivot down and around the body member. The side walls may be connected by an upper wall comprising the release member already described above. In these and other embodiments, the release member may be formed in the upper wall of the lever member by a living hinge. The release member may therefore be operable to move towards the body member independently of the side walls. In at least some such embodiments, the lever member may comprise one or more side walls linked by a bridge section that passes across the body member. Furthermore, the release member may be arranged to extend over the bridge section, such that the release member must be operated in an initial stage to move towards the body member before contacting the bridge section to start transferring a force to the lever member. In a further stage, further movement of the release member towards the body member also drives movement of the rearward portion of the lever member towards the body member. This provides for a particularly intuitive mode of two-stage operation, as a user cannot easily access the bridge section to apply a force without using the release member that extends over the bridge section. This means that the release member is intuitively operated first to actively release the disconnection member when it is locked in the first position.

In such embodiments, the device may comprise a body member to which the lever member is pivotally mounted. The lever member may comprise a disconnecting portion arranged to move at least partially along an axis of the fluid transfer tip away from the body member, thereby acting on the hub to advance it along the fluid transfer tip. In such embodiments, the lever member may further comprise a rearward portion extending away from the disconnecting portion, the rearward portion arranged to interact with the body member such that the lever member is locked in the first position, as described generally above. In such embodiments, the device may further comprise a release member arranged to deform the rearward portion around the body member so as to release the lever member from the first position. This may be achieved, for example, by deforming one or more walls of the lever member outward relative to the body member. In a set of embodiments, the release member comprises deformable side legs which extend towards and rest on an outer surface of the body member and arranged such that, as the release member is pressed down towards the body member, the side legs are forced to splay outwards around the body member to contact one or more walls of the lever member and further operation of the release member thereby drives pivotal movement of the lever member. During such pivotal movement, the rearward portion may move towards the body member while the disconnecting portion moves away from the body member and along the fluid transfer tip. Optionally, the device may include a latch on side walls of the lever member which interacts with the side legs of the release member to pull the lever member back up.

In one or more other embodiments, the disconnection member is a sliding member. The sliding member may be mounted to slide along a body member of the device from a first position over the body member to a second position over the fluid transfer tip. As described above, the sliding member may be integrated with the collar. Movement of the sliding member may cause the second segment to move as described hereinabove. In embodiments where the second segment comprises a threaded portion, the threaded portion may act as a disconnection portion that advances along the fluid transfer tip.

The first aspect of the present invention extends to a system comprising a hub and medical device connector as described herein, wherein the hub comprises the corresponding engagement feature, preferably arranged at a first end of the hub, for engaging with the engagement feature of the second segment. There has already been described a second aspect of the present invention wherein engagement between the hub and the collar is optional but the collar comprises a deceleration feature. The Applicant has now appreciated that even in the absence of a deceleration feature, or any engagement features, a separable collar operated by the disconnection member may be desirable as the collar can prevent misconnections at least in its closed configuration. Thus, when viewed from a third aspect, the present invention provides a medical device connector for connecting, in use, to a hub, the medical device connector comprising: a fluid transfer tip comprising a tapered surface for creating a friction fitting between the fluid transfer tip and a hub connected to the fluid transfer tip in use; a separable collar, comprising at least a first arcuate segment and a second arcuate segment, which extend at least partially around the fluid transfer tip, wherein at least one of the first and second segments is moveably mounted relative to the fluid transfer tip, and wherein each of the first and second segments comprises a surface facing the fluid transfer tip that is devoid of engagement features; the separable collar having a closed configuration in which the second segment is positioned relative to the fluid transfer tip so as to extend around a hub connected to the tip in use, and an open configuration in which the first and/or second segment is moved relative to the fluid transfer tip; and a moveable disconnection member arranged such that movement of the disconnection member moves the first and/or second segment of the separable collar from a first position, corresponding to the closed configuration of the separable collar, to a second position, corresponding to the open configuration of the separable collar, and wherein movement of the disconnection member also releases the friction fitting between the fluid transfer tip and the hub by advancing the hub along the tapered surface of the fluid transfer tip.

It will be understood that the surface facing the fluid transfer tip being devoid of engagement features means that the first and second segments of the separable collar do not include any threaded portion, or other engagement feature such as a latch or otherwise. The separable collar may therefore be viewed as a passive “dummy” collar that provides a visual indicator to users to avoid confusion and misconnections.

Preferably the first segment is arranged in a fixed position relative to the fluid transfer tip and the second segment is moveably mounted relative to the fluid transfer tip and the first segment, as is described above.

A medical device connector as described herein may take the form of a fluid transfer connector or a fluid transfer device, such as a syringe. The medical device connector may be a pre-filled syringe. Instead of a hub being connected to the fluid transfer tip in use, a cap may be connected to seal the syringe. Furthermore, the Applicant has appreciated that it can be particularly convenient to connect a disinfecting cap to the fluid transfer tip, not only to seal a pre-filled syringe, but more generally to provide a medical device connector that can be supplied to a user with the disinfecting cap pre-connected rather than loose in a kit. This can help to promote aseptic procedures.

Thus, in one or more embodiments of any aspect of the invention, there is provided a system comprising a medical device connector and a disinfecting cap, wherein the disinfecting cap comprises: a connection portion comprising a tapered internal surface and an external engagement feature for connecting the cap to the medical device connector; and wherein the medical device connector comprises: a fluid transfer tip comprising a tapered surface for creating a friction fitting between the fluid transfer tip and the tapered internal surface when the cap is connected to the medical device connector; and a separable collar, comprising at least a first arcuate segment and a second arcuate segment, which extend at least partially around the fluid transfer tip, wherein at least one of the first and second segments comprises an internal engagement feature for engaging with the external engagement feature of the cap connected to the fluid transfer tip in use.

The collar may be manually separable, but preferably the medical device connector further comprises a moveable disconnection member arranged such that movement of the disconnection member moves the first and/or second segment of the separable collar from a first position, corresponding to a closed configuration of the separable collar (in which least one of the first and second segments is positioned relative to the fluid transfer tip so as to extend around a cap connected to the tip in use and to engage with the cap by engagement between the internal and external engagement features) to a second position, corresponding to the open configuration of the separable collar (in which least one of the first and second segments is moved relative to the fluid transfer tip, so as to allow disengagement of the internal and external engagement features), and wherein movement of the disconnection member also releases the friction fitting between the fluid transfer tip and the cap by advancing the cap along the tapered surface of the fluid transfer tip.

In such a system, the medical device connector may further comprise any of the features already described above. For example, the first segment may be arranged in a fixed position relative to the fluid transfer tip and the second segment may be moveably mounted relative to the fluid transfer tip and the first segment. The medical device connector may be a pre-filled syringe.

Such a disinfecting cap is also considered novel in its own right, and thus when viewed from a further aspect the present invention provides a disinfecting cap comprising a connection portion comprising a tapered internal surface and an external engagement feature for connecting the cap to a medical device connector. The external engagement feature may comprise one or more of: a circumferential flange, a screw thread, a spline, or a latch. One or more such external engagement features may be present. In addition to the connection portion, the disinfecting cap may comprise any of the standard feature of a disinfecting cap, such as a chamber containing disinfecting liquid.

### BRIEF DESCRIPTION OF THE DRAWINGS

Some embodiments of the present invention will now be described, by way of example only, and with reference to the accompanying drawings, in which:

FIG. 1 shows a perspective view of a medical device connector in the form of a syringe according to a first embodiment;

FIGS. 2A-2E show operation of the medical device connector seen in FIG. 1;

FIG. 3 shows a perspective view of the lever member and release member as seen in FIG. 1;

FIG. 4 shows a perspective view of the fluid chamber seen in FIG. 1;

FIG. 5 is a perspective view illustrating assembly of the syringe seen in FIG. 1;

FIG. 6 is a rear view illustrating the assembly of the syringe seen in FIG. 1;

FIGS. 7A-7B schematically show an NR Fit hub;

FIGS. 8A-8B schematically show a Luer Fit hub;

FIGS. 9A-9B demonstrate how misconnection is prevented with the syringe seen in FIG. 1;

FIGS. 10A-10D illustrate a second embodiment of a medical device connector in the form of a syringe;

FIGS. 11A-11B illustrate a third embodiment of a medical device connector in the form of a syringe;

FIGS. 12A-12B show the disconnection member seen in FIGS. 11A-11B;

FIGS. 13A-13C illustrate a fourth embodiment of a medical device connector in the form of a syringe;

FIGS. 14A-14B illustrate a fifth embodiment of a medical device connector in the form of a fluid transfer connector;

FIGS. 15A-15B illustrate a sixth embodiment of a medical device connector in the form of a Luer fluid transfer connector;

FIGS. 16A-16B illustrate a seventh embodiment of a medical device connector in the form of a fluid transfer connector;

FIGS. 17A-17B illustrate an eighth embodiment of a medical device connector in the form of a fluid transfer connector;

FIG. 18 shows a close-up view of a separable collar, wherein the first segment comprises slots for receiving the disconnection portion of the disconnection member;

FIG. 19 shows a close-up view of a separable collar, wherein the first segment is separated into spaced parts to receive the disconnection portion of the disconnection member;

FIG. 20 shows a close-up perspective view focusing on the separable collar with a continuous threaded portion;

FIG. 21 shows a close-up perspective view focusing on an alternative embodiment wherein the collar comprises a discontinuous threaded portion;

FIGS. 22A-22B show a medical device connector comprising a deceleration feature which comprises three protrusions;

FIG. 23 shows an alternative medical device connector comprising a deceleration feature which comprises only a single protrusion;

FIG. 24 shows a perspective view of a disinfecting cap;

FIGS. 25A-25C show a disinfecting cap attached to a pre-filled syringe, an underside view of the disconnection member, and operation of the disconnection member to release the disinfecting cap from the prefilled syringe;

FIGS. 26A-26B show perspective views of a further embodiment of a medical device connector in the form of a fluid transfer connector, with the first and second segments latched together and separated;

FIGS. 27A-27B show perspective views of a further embodiment of a medical device connector in the form of a fluid transfer connector; and

FIGS. 28A-28C show perspective views of a further embodiment of a medical device connector in the form of a syringe.

### DETAILED DESCRIPTION

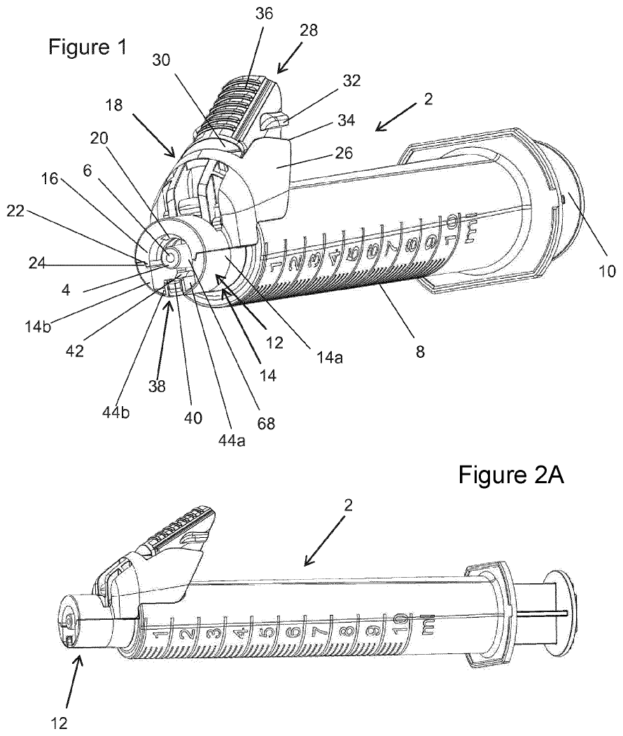

FIG. 1 shows a medical device connector in the form of a syringe 2 in accordance with a first embodiment of the invention. The syringe 2 comprises a fluid transfer tip 4 comprising a tapered surface with a fluid channel 6 extending therethough to a fluid chamber 8. The syringe 2 is provided with a plunger 10 for either drawing fluid in, or expelling fluid from, the fluid chamber 8. The syringe 2 further comprises a separable collar 12 which surrounds the fluid transfer tip 4. The separable collar 12 comprises a first arcuate segment 14 which extends from the fluid chamber 8, and is fixed relative to the fluid transfer tip 4. The separable collar 12 further comprises a second arcuate segment 16. In this embodiment, the first and second arcuate segments 14, 16 each has an outer profile that is curved, so that the profile of the collar 12 is circular i.e. at a fixed radius from the fluid transfer tip 4 in a symmetrical arrangement. The second arcuate segment 16 extends from a disconnection member in the form of a lever member 18 which is pivotally mounted to the fluid chamber 8. Movement of the lever member 18 thereby results in movement of the second segment 16, e.g. pivotal movement. The second segment 16 comprises engagement features in the form of a threaded portion 20, arranged on an inside surface thereof, which project towards the fluid transfer tip 4.

The second segment 16 comprises a tongue 22 at each edge of the arcuate segment 16 and the first segment 14 comprises a receiving portion 24 for receiving the tongue 22. FIG. 1 shows the separable collar 12 in the closed configuration. Here it can be seen that both the first segment 14 and second segment 16 extend around the fluid transfer tip 4. Furthermore, the tongue 22 and receiving portion 24 form a completely closed collar 12 around the fluid transfer tip 4.

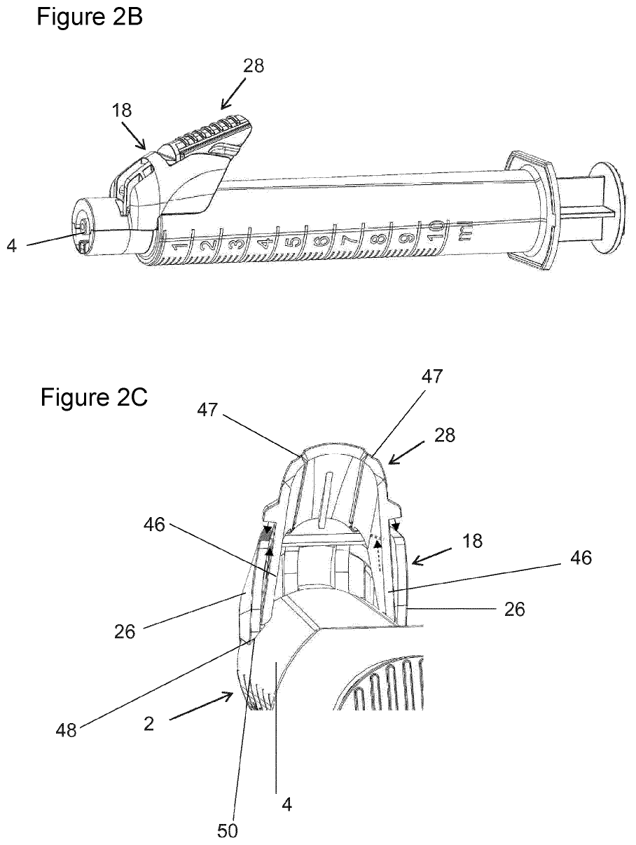

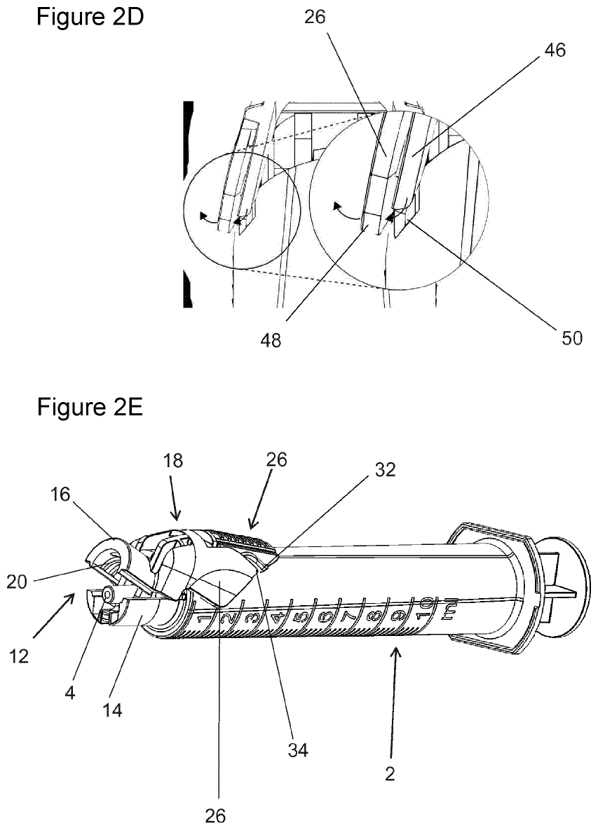

The lever member 18 of this particular embodiment is formed in a three-dimensional shell shape which comprises side walls 26 which engage with locking ledges (not shown in this Figure) on the fluid transfer chamber 4 so as to inhibit movement of the lever member 18. A release member 28 is provided which is connected to the lever member 18 via a living hinge 30. The release member 28 is therefore able to pivot relative to the lever member 18. The release member 28 functions to release the side walls 26 of the lever member 18 from the locking ledges on the fluid transfer chamber 4, as will be described later with reference to FIGS. 2A-E. The release member 28 also comprises transfer ledges 32 which engage with an upper surface 34 of the side walls 26 of the lever member 18, when the release member 28 is pivoted by a sufficient amount. This is also described later with reference to FIGS. 2A-2E. The release member 28 is further provided with ridges 36, on a top surface thereof, to provide improved grip for a user when operating the release member 28.

The syringe 2 further comprises a deceleration feature 38 which comprises an arm 40 and a protrusion 42 extending therefrom towards the fluid transfer tip 4. The deceleration feature 38 divides the first segment 14 of the collar 12 into two portions: a first portion 14a and a second portion 14b. The first portion 14a and second portion 14b each comprises a slot 44a, 44b respectively for receiving a forked disconnection portion (not visible in this Figure) of the lever member 18.

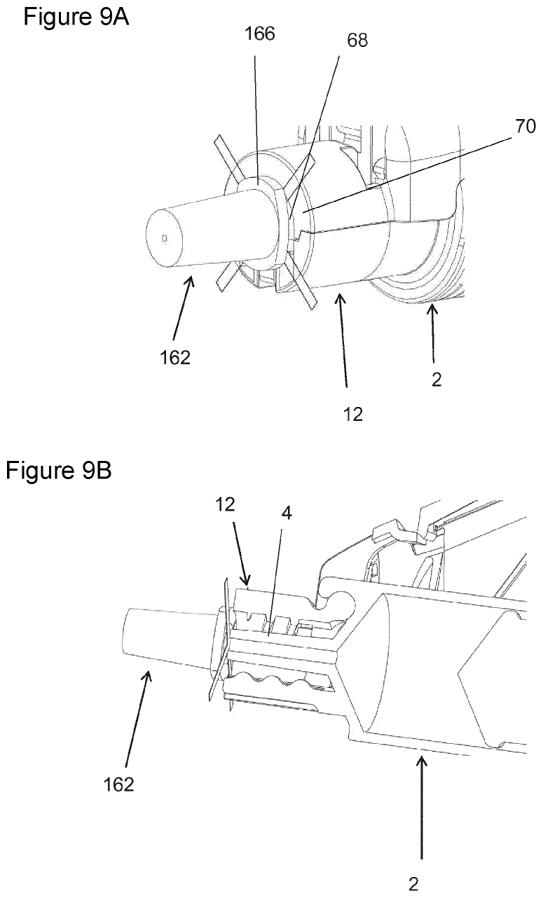

With the separable collar 12 in the closed configuration as seen in FIG. 1, the separable collar 12 defines a space 68 extending around the fluid transfer tip 4. As the first segment 14 is fixed relative to the fluid transfer tip 4, and the second segment 16 is also held in a fixed position, due to the lever member 18 being locked in position, in the closed configuration, the space 68 defined by the collar 12 cannot be changed. The collar 12 is designed such that the space 68 is capable of accommodating a specific type of collar. As will be appreciated by those skilled in the art, when a user offers a correct hub to the syringe 2, its dimensions will allow it to connect on to the fluid transfer hub 4 and create a friction fit, and also allow it to pass into the space 68 defined by the collar 12 and become engaged by the threaded portion 20 on the second segment 16. However, in the situation where a user offers up an incorrect hub to the syringe, the hub will be prevented from creating a friction fit with the fluid transfer tip 4, and also from being engaged by the threaded portion 20, as the space 68, will not be sufficient to accommodate the hub. In this instance the hub will simply abut against the outer, end surface of the collar 12 and therefore be prevented from being attached to the syringe 2. This is demonstrated in FIGS. 9A and 9B. Accordingly, the syringe 2 in accordance with embodiments of the present invention will assist in preventing misconnections of hubs.

Furthermore, the syringe 2 may also prevent misconnection of an incorrect hub even when the collar 12 is in an open configuration, i.e. when the second segment 16 is pivoted away from the fluid transfer tip 4. In this instance, whilst the second segment 16 may be pivoted away and thus it does not provide the specific spacing around the fluid transfer tip 4 which is suitable for a specific type of hub, the first segment 14 remains fixed relative to the fluid transfer tip 4 and thus still provides a fixed space between the first segment 14 and the fluid transfer tip 14. Accordingly, whilst the entire collar 12 may not provide the defined space 68 as seen in FIG. 1, if the first segment 14 has a sufficient extent, and is sufficiently rigid, the space which it defines on its own, irrespective of the second segment 16, may be sufficient to prevent the misconnection of an incorrect hub.

FIGS. 2A-2E show operation of the syringe 2. FIG. 2A shows the syringe 2 with the separable collar 12 in the closed configuration. Whilst a hub is not shown connected to the fluid transfer tip 4 of the syringe 2 in this Figure, it will be appreciated that a hub may be connected to the syringe 2 in use.

FIG. 2B shows the first stage required when a user wishes to disconnect a hub from the fluid transfer tip 4. First a user must depress the release member 28 so as to release the lever member 18 from its locked position. This process is seen in more detail in FIG. 2C, which shows a close-up view from the rear of the syringe 2 focusing on the rear of the release member 28 and lever member 18. As can be seen in FIG. 2C, the release member 28 comprises side legs 46 which extend towards and rest on the outer surface of the fluid chamber 8. As the release member 28 is depressed, the side legs 46 splay outwards towards the side walls 26 of the lever member 18. The splaying of the side legs 46, in this particular embodiment, is made possible through the provision of two integral hinge lines 47, i.e. living hinges, provided on the release member 428. These hinge lines 47 permit the side legs 46 to splay outwards and help to avoid, or at least minimize, any deformation of the side legs 46 themselves. The integral hinge lines 47 are provided by a thinner portion of the material of the release member 428 along the hinge lines 47. This thinner portion may be achieved directly, for example during an injection molding process. As shown in this Figure, the bottom edges 48 of the side walls 26 are locked into locking ledges 50 provided on the fluid chamber 8, thereby preventing movement of the lever member 18. As the release member 18 is depressed, the side legs 46 splay outwards towards the side walls 26 of the lever member 18.

Once the release member 18 has been depressed by a sufficient amount, the side legs 46 will force the side walls 26 outwards. FIG. 2D shows the point at which the side legs 46 force the side walls 26 outwards by a sufficient amount such that the bottom edge 48 thereof is no longer in locked in place by the locking ledge 50. At this point, the lever member 18 can be pivoted.

Once the lever member 18 has been released from the locking ledge 50 as described above, depression of the release member 28 may further act to pivot the lever member 18. This is achieved by the transfer ledge 32 acting on the upper edge 34 of the side walls 26 of the lever member 18. As the release member 28 is pressed, the transfer ledge 32 acts on the upper edge 34 and forces the lever member 18 to pivot. This can be seen in FIG. 2E. As the lever member 18 is pivoted, the second segment 16 is pivoted away from the fluid transfer tip 4 so that the thread 20 no longer engages with a hub attached to the syringe 2. As seen in FIG. 2E, the first segment 14 remains fixed and does not move as the lever member 18 is pivoted.

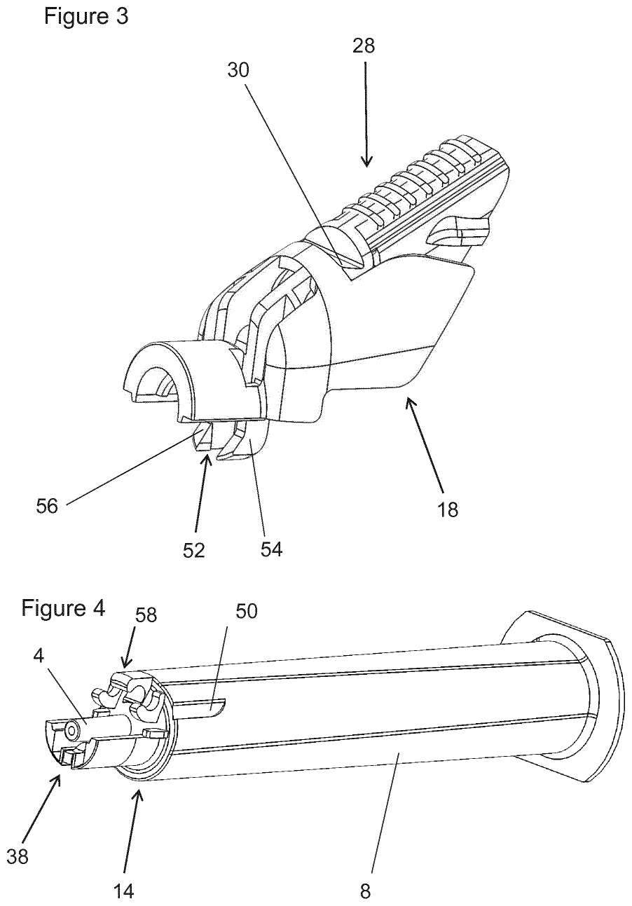

Although a hub is not seen in FIGS. 2A-2E, it will be understood that pivotal movement of the lever member 18 also acts to advance any connected hub along the tapered surface of the fluid transfer tip 4, because the lever member 18 includes a disconnection portion arranged to push off the hub, as described further below with reference to FIG. 3.

In the embodiment described above, once a user removes the applied force, e.g. releases their thumb, from the release member 28, the release member 28 and lever member 18 may automatically move back to the position seen in FIG. 2A. This may be achieved by an integral resilient bias provided by both the release member 28 and the lever member 18. As the side legs 46 of the release member 28, and the side walls 26 of the lever member 18, flex outwards as described above, the material from which these parts are made may have inherent resilience which may cause the release member 28 and lever member 18 to return back to the shape and configuration seen in FIG. 2A. However, as will be appreciated, in addition or alternatively, at least the lever member 18, and optionally also the release member 28, may be provided with a separate resilient bias, e.g. provided by a spring member in the form of a leaf spring.

FIG. 3 shows a perspective view of the lever member 18 and release member 28 as seen in FIG. 1, showing the living hinge 30 between the lever member 18 and the release member 28. In this particular embodiment, the disconnection member 18, 28 is a separate component which is attached to the fluid chamber 8 of the syringe 2 seen in FIG. 1. Also visible in this Figure is the disconnection portion which is provided by a forked section 52. Each arm 54, 56 of the forked section 52 extends either side of the fluid transfer tip 4 when the lever member 18 is attached to the fluid chamber 8. When the lever member 18 is pivoted, each arm 54, 56 of the forked section 52 extends into the slots 44a, 44b. The provision of the slots 44a, 44b ensures that the arms 54, 56 of the forked section 52 can be longer than they otherwise would be if there were no slots 44a, 44b, present. This means that, as the lever member 18 is pivoted, the arms 54, 56 extend further along the fluid transfer tip 4 and thus act to advance a hub attached thereto across a longer distance thereby improving the ability for the lever member 18 to disconnect the hub from the fluid transfer tip 4.

FIG. 4 shows a perspective view of the fluid chamber 8 without the disconnection member (i.e. lever member 18 and associated release member 28) attached. As can be seen, the fluid transfer tip 4, the first segment 14 and the deceleration feature 38 all extend directly from the fluid chamber 8, and in this particular embodiment all of these features are provided by an integrally molded component. Additionally, the locking ledge 50 is also integrally provided with the fluid chamber 8. In this particular embodiment, a second locking ledge 50 is provided on the opposite side of the fluid chamber 8, however it cannot be seen in this Figure. It will be appreciated, however, that it may not be necessary to provide two locking ledges 50 and instead one may suffice to hold the lever member 18 in position. The fluid chamber 18 also comprises hooks 58 for receiving an axle portion of the lever member 18 in order to pivotally mount the lever member 18 to the fluid chamber 8.

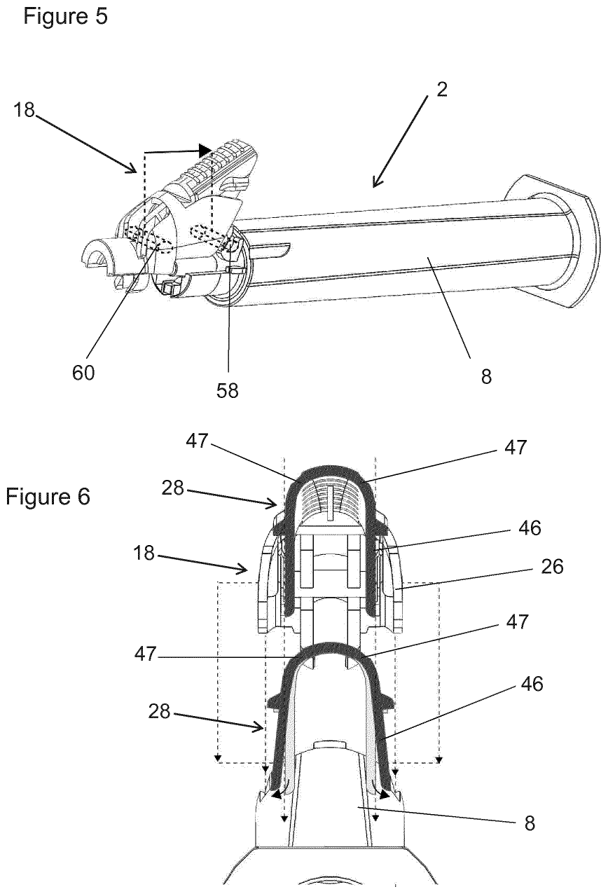

FIG. 5 shows an isometric view illustrating how the lever member 18 can be attached to the fluid chamber 8 to provide the complete syringe 2 as seen in FIG. 1. The lever member 18 may be slid into place such that an axle 60 provided thereon is engaged by the hooks 58 provided on the fluid chamber 8. Once attached to the fluid chamber 8, the lever member 18 may be selectively detached by applying an appropriate force to the lever member 18 to separate the axle 60 from the hooks 58. This may, for example permit the lever member 18 to be disposed of separately from the fluid chamber 8.

FIG. 6 shows that, prior to being mounted to the fluid chamber 8, the side legs 46 of the release member 28, and also the side walls 26 of the lever member 18, extend vertically downwards. As the lever member 18, and thus the release member 28, are mounted to the fluid chamber 8, as depicted by the bottom half of FIG. 6, the side legs 46 of the release member 28, and also the side walls 26 of the lever member 18 (not shown in this part of the Figure), are splayed outwards due to the curved shape of the fluid chamber 8. As can be seen in the lower part of this Figure, the splaying of the side legs 46, at least, is made possible due to the hinge lines 47 which are integrally provided as part of the release member 28, i.e. a living hinge. These hinge lines 47 allow the side legs 46 to splay outwards with minimum, or no, deformation of the side legs 47 themselves. As will be appreciated by those skilled in the art, this may help to avoid unnecessary fatigue on the side legs 46 and thus may ensure the proper function and avoid failure of the mechanism. Whilst not show, the lever member 18 may also be provided with similar such hinge lines to allow its side walls 26 to splay outwards.

The medical device connectors described herein, such as the syringe 2 seen in FIGS. 1-6, are designed to meet at least one of the ISO 80369 series of small-bore connector standards for fluid connectors in healthcare applications. In some embodiments, the fluid transfer tip 4 and separable collar 12 together provide a Luer connector part that is compliant with ISO 80369-7 for connections in intravascular applications or hypodermic connections in hypodermic applications of medical devices and accessories. In some embodiments, the fluid transfer tip 4 and separable collar 12 together provide an ENFit connector part. An ENFit connector part is compliant with ISO 80369-3 for connections on enteral medical devices and accessories. In some embodiments, the fluid transfer tip 4 and separable collar 12 together provide an NRFit connector part. An NRFit connector part is compliant with ISO 80369-6 for connections in neuraxial applications.

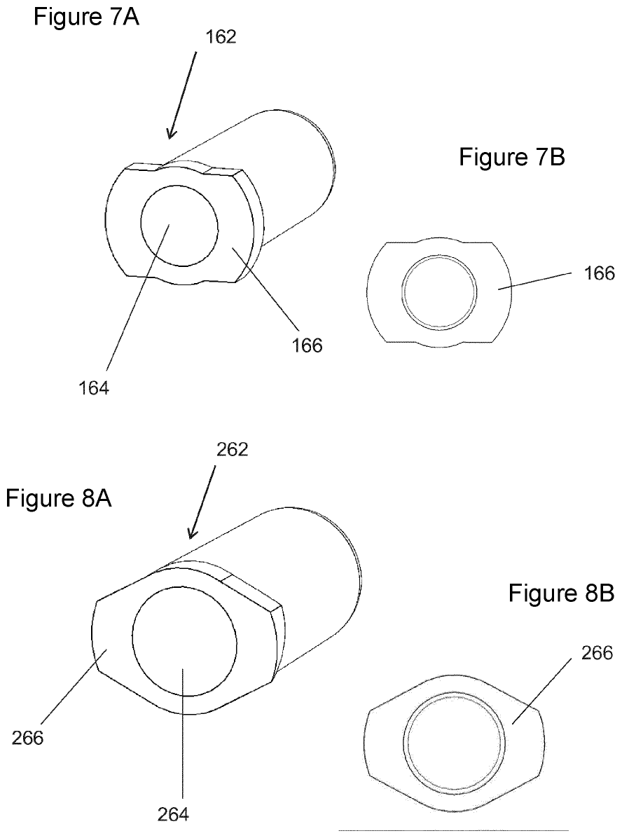

FIGS. 7A and 7B show a perspective view and a rear view, respectively, of an NRFit hub 162. The hub 162 comprises a fluid channel 164 passing therethrough to permit the passage of fluid. At a rear end thereof the hub 162 comprises a specifically shaped flange 166. This shape can be seen more clearly in the rear view seen in FIG. 7B. The flange shape 166 is specific to an NRFit hub and has dimensions which meet the ISO 80369-6 standard.

FIGS. 8A and 8B show a perspective view and a rear view, respectively of a Luer hub 262. The hub 262 comprises a fluid channel 264 passing therethrough to permit the passage of fluid. At a rear end thereof the hub 262 comprises a specifically shaped flange 266. This shape can be seen more clearly in the rear view seen in FIG. 8B. The flange shape 266 is specific to a Luer hub and has dimensions which meet the ISO 80369-7 standard.

As can be seen by comparison of FIGS. 7A and 7B with FIGS. 8A and 8B, each of the hubs 162, 262 comprises a flange 166, 266 which is shaped differently and has different dimensions. Accordingly, as will be appreciated by those skilled in the art, the separable collar 12 provided on the syringe 2 is dimensioned so as to permit only one specific type of hub to be attached to the fluid transfer tip 4 of the syringe 2. This is illustrated in FIGS. 9A and 9B. FIG. 9A shows a close-up perspective view of the front end of the syringe 2 as seen in FIG. 1. Shown is an NRFit hub 166 which is prevented from being attached to the syringe 2 which is designed for use with an alternative type of hub, e.g. a Luer hub. As the hub 162 is brought towards the fluid transfer tip 4 (not visible in FIG. 9A), the space 68 provided between the separable collar 12 and the fluid transfer tip 4 is too small to allow the flange 166 in. As a result, the flange 166 simply abuts against an end face 70 of the separable collar 12 and is prevented from being connected to the syringe 2. This is further shown in FIG. 9B, which shows a cross-sectional view of the syringe 2 and focusses on the forward end thereof. As is apparent from this Figure, in this particular embodiment the fluid transfer tip 4 does not extend any further forward than the separable collar 12 and, as a result, when the hub 162 is stopped by the separable collar 12 it is also prevented from connecting to the fluid transfer tip 4 in any way. This ensures that no confusion arises regarding connection, for example by avoiding the hub 162 from partially connecting to the fluid transfer tip 4 only. The fact that the hub 162 is prevented from being connected is illustrated by the “X” across the front of the fluid transfer tip 4.

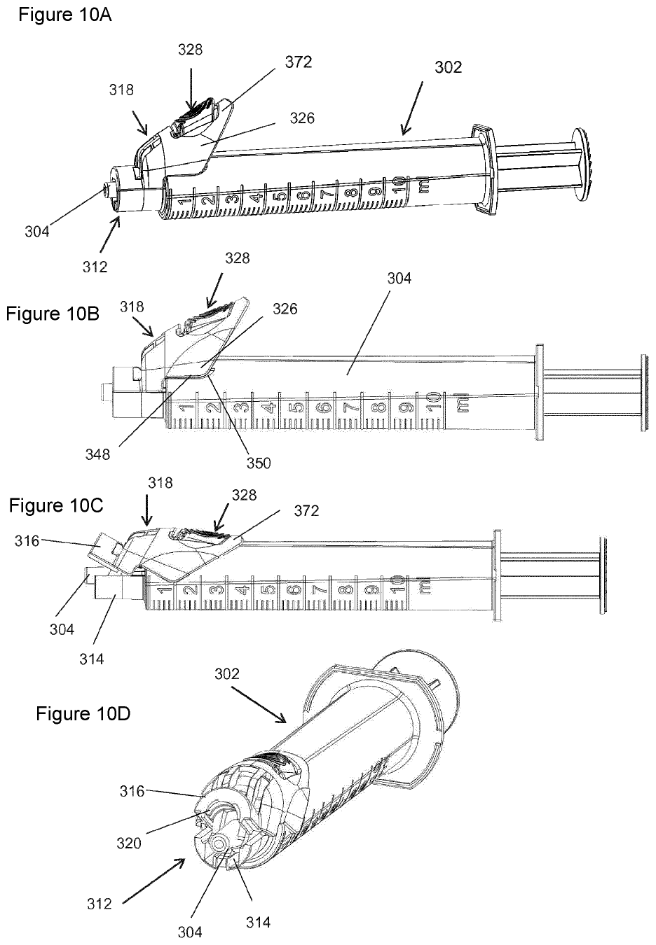

FIGS. 10A-10D show a further embodiment of a medical device connector in the form of a syringe 302 and also illustrate the function of the syringe 302. FIG. 10A shows a perspective view of the syringe 302. The syringe 302 is essentially the same as the syringe 2 seen in earlier Figures, except that it comprises a different lever member 318 and release member 328 acting together as the disconnection member. The lever member 318 comprises a bridge section 376 at the rear of the lever member 318 which joins the side walls 326 of the lever member 318. This bridge section 376 serves to increase the rigidity of the lever member 318 and also serves as a place for a user to apply a force to the lever 318 in order to depress it.

Operation of the syringe 302, specifically the lever member 318 and release member 328, will now be described with reference to FIGS. 10A to 10D. In FIG. 10A the separable collar 312 is held in the closed configuration, i.e. it surrounds the fluid transfer tip 304. When a user wishes to disconnect a hub from the fluid transfer tip 304, for example a hub of the type seen in FIGS. 7A and 8A, they must first press the release member 328 in order to unlock the lever member 318. Depression of the release member 328 can be seen in FIG. 10B. As shown, the release member 328 can be depressed downwards towards the fluid chamber 304. At the position seen in FIG. 10B, the release member 328 functions to push the side walls 326, of the lever member 318, outwards so that the bottom edge 348 thereof is no longer engaged with the locking ledge 350. In this regard, the release member 328 functions in the same manner as the release member 28 seen in the first described embodiment, specifically with regard to FIGS. 2C and 2D.

Once the lever member 318 has been unlocked as described above, the lever member 318 may then be pivoted. This can be seen in FIG. 10C. This embodiment differs, however, in that the release member 328 does not comprise any transfer ledges 32 to act on the lever member 318. Instead, a user will need to apply a force to the bridge section 372 in order to pivot the lever member 318. Due to the arrangement of the release member 328 within the lever member 318, following depression of the release member 328, a user's thumb, for example, will naturally rest on the bridge section 372 and thus a user can easily apply a force to the bridge section 372 following unlocking of the lever member 318. As seen in FIG. 10C, once the lever member 318 has been pivoted the second segment 316 is also pivoted away from the fluid transfer tip 304, into an open configuration, so as to release any engagement between the engagement features (not visible in this Figure) and a hub attached to the fluid transfer tip 304.

FIG. 10D shows a perspective view of the front of the syringe 302 with the lever member 318 positioned such that the collar 312 is in the open configuration, with the second segment 316 pivoted away from the stationary first segment 314. It is clear from this Figure that the other features of the syringe 302 are identical to those of the syringe 2 described above. The second segment 316 comprises a threaded portion 320 that is pivoted out of engagement with a corresponding engagement feature of a hub (not shown) connected to the tip 304 in use.

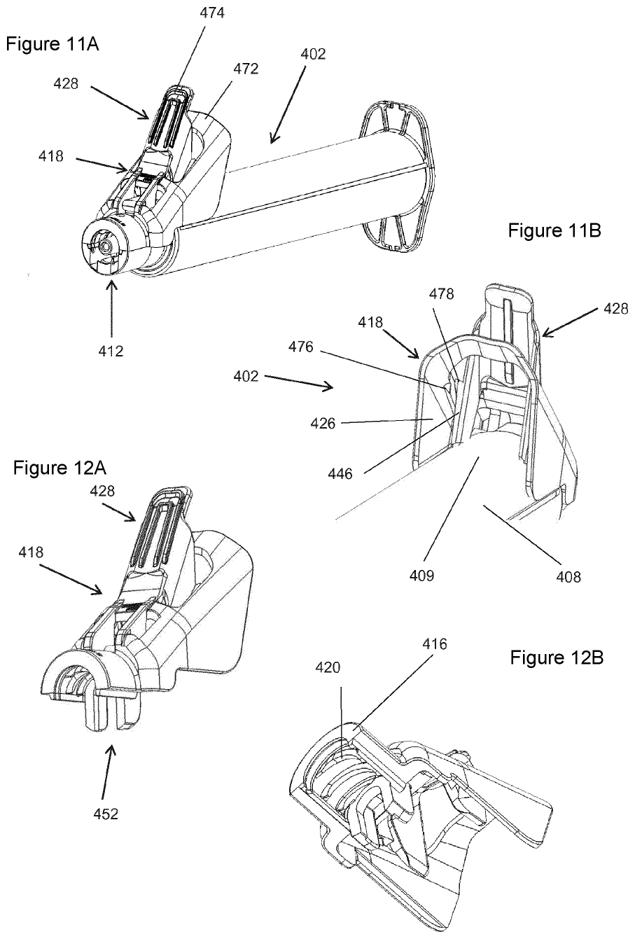

FIG. 11A shows a perspective view of a medical device connector in the form of a syringe 402 in accordance with a further embodiment of the present invention. The syringe 402 is essentially the same as the syringe 302 seen in FIG. 10A, except that the release member 428 takes a slightly different form. The release member 428 comprises a transfer portion 474 extending from the rear of the release member 428. Similarly to the transfer ledges 32 of the first embodiment seen in FIG. 1, the transfer portion 474 acts to transfer a force from the release member 428 to the lever member 418 as the release member 428 is depressed. Unlike the first embodiment wherein the transfer ledges 32 act on the top edge 34 of the sidewalls 26 of the lever 18, in this embodiment, the transfer portion 474 acts on the bridge portion 472 of the lever member 418 in order to pivot the lever member 418. The Applicant has recognized that the arrangement of the transfer portion 474 at the rear end of the release member 428 helps to ensure that the syringe 402, specifically the lever member 418 and release member 428, is intuitive to use. A user simply applies a force with, for example, their thumb to the release member 428 to both release the lever member 418 from its first, locked position, and to drive the lever member 418 into its second position, without having to adjust their grip on the syringe 402.

FIG. 11b shows a perspective view of the syringe 402 when viewed from the rear, with the disconnection member comprising the lever member 418 and release member 428 partially assembled to the fluid chamber 408. As can be seen in this view, the lever member 418 comprises a chamfered ledge 476 on the internal surface of each side wall 426. The release member 428 comprises a chamfered hook 478 on the outside facing surface of each side leg 446. During manufacture or assembly of the syringe 402, the lever member 418 and release member 428 are initially pivotally mounted to the fluid chamber 408 so as to form the arrangement as seen in FIG. 11b. Once pivotally mounted, a second stage (not illustrated) follows in which the release member 428 is depressed such that side legs 446 begin to splay around the curved upper face 409 of the fluid chamber 408. As this initial splaying of the side legs 446 occurs, the hooks 478 slip past the ledges 476, on the side walls 426 of the lever member 418. Once the hooks 478 have slipped past the ledges 476, due to the shaping of the hooks 478 and ledges 476, and once the force tending to move the release member 428 downwards is released, the release member 428 will move back upwards to a position wherein the hooks 478 and ledges 476 engage with one another and thus prevent further movement of the release member 428. As will be appreciated, the release member 428, specifically at least the side legs 446 thereof, is/are designed such that in this position wherein the hooks 478 engage with the ledges 476 the side legs 446 are at least partially splayed and therefore provide a resilient force driving the release member 428, and holding it, in this position. Once in this ‘default’ position, the syringe 402 may then be ready for operation. This stage of pushing the release member 428 into a position whereby the hooks 478 and ledges 476 are engaged is ideally carried out during manufacture or assembly of the syringe 402, however this stage may instead be carried out by a user prior to use of the syringe 402.

Starting from the default position described above, a user may apply a force to the release member 428 in order to release the lever member 418 from its first, locked position, and thereby pivot the lever member 418 into a second position, corresponding to the collar 412 being in an open configuration. This may be, for example to release a hub from the syringe 402. Following this operation, a user may subsequently release the force applied to the release member 428, e.g. by removing their thumb, or other finger from the release member 428. When a user releases the force on the release member 428, due to the resilient bias provided as a result of the side legs 446 splaying, the release member 428 will tend to move towards the position seen in FIG. 11A. As the release member 428 moves towards this position, the hook 478, provided on the release member 428, will pull on the ledge 476, provided on the lever member 418, and thereby act to pull the lever member 418 into the position seen in FIG. 11A. As will be appreciated, the release member 428 thereby provides a resilient bias to bring the lever member 418 into the closed configuration. This resilient bias may be in addition to a resilient bias provided by the lever member 418, e.g. by deformation of the side walls 426 of the lever member 418. The Applicant has recognized that this double resilient bias may ensure that the separable collar 412 is held securely in the closed position seen in FIG. 11A.

The Applicant has recognized that the arrangement described above comprising the ledges 476 and hooks 478 which act to link the lever member 418 and release member 428 in the manner described may be applied to any embodiments comprising a pivotally mounted lever member and associated release member.

FIG. 12A shows a perspective view of the lever member 418 and associated release member 428 as seen in FIG. 11B. Similarly to earlier embodiments, the lever member 418 comprises a disconnection portion provided by a forked section 452. FIG. 12B shows a perspective view when viewed from underneath thereby showing the engagement feature in the form of a continuous threaded portion 420 provided on the second segment 416.

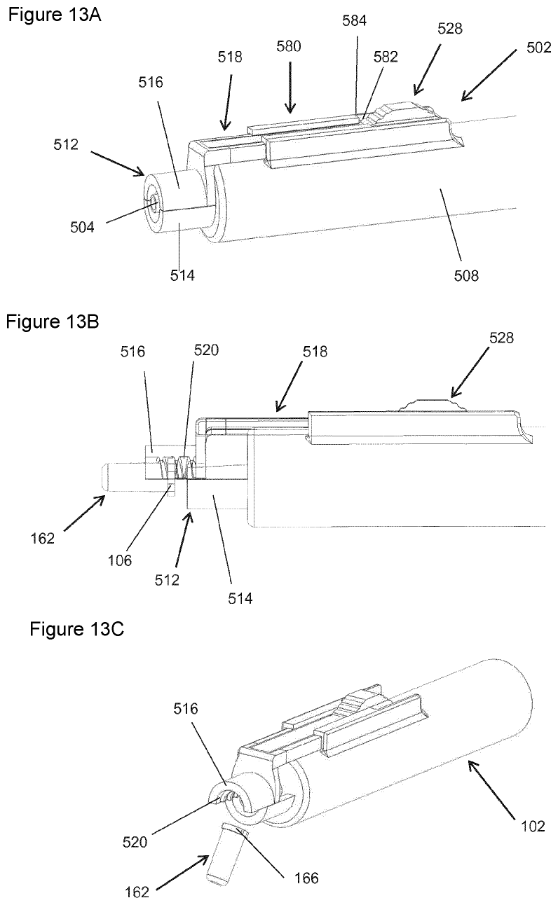

FIG. 13A shows an alternative embodiment of a medical device connector in the form of a syringe 502 with a different type of disconnection member to those discussed above. Similarly to the first embodiment described above, the syringe 502 comprises a fluid transfer tip 504 which extends into a fluid chamber 508. A plunger (not shown) may be provided in order to expel a fluid from the fluid chamber 508 or to draw a fluid into the fluid chamber 508. The syringe 502 further comprises a separable collar 512 comprising a first arcuate segment 514 which extends from the fluid chamber 508 and is thereby fixed relative to the fluid transfer tip 504. A second arcuate segment 516 is also provided. In this embodiment, instead of a lever member, the disconnection member is provided by a sliding member 518 which is arranged in a guide 580 which is arranged on the fluid chamber 508. The guide 580 acts to restrict the movement of the sliding member 518 such that it only moves linearly with respect to the fluid chamber 508 and the fluid transfer tip 504. The second segment 516 is directly attached to the sliding member 518 such that movement of the sliding member 518 directly results in movement of the second segment 516 with respect to the fluid transfer tip 504. The second segment 516 comprises a threaded portion 520 on its internal surface, as seen in FIGS. 13B and 13C.

The syringe further comprises a release member 528 which extends from a rear portion of the sliding member 518. The release member 528 comprises a front edge 582 that, at least in the position seen in FIG. 13A, engages with a locking edge 584 on the guide 580. This engagement prevents the sliding member 518 from moving relative to the guide 580, and thereby holds the first segment 516 in a fixed position. The sliding member 518 and release member 528 are biased upwards, away from the fluid chamber 508, such that the front edge 582 of the release member 528 engages with the locking edge 584 on the guide 580. This may be achieved, for example, by providing the sliding member 518 and release member 528 as a curved element which curves upwards, away from the fluid chamber 504.

When a user wishes to disengage a hub (not shown in FIG. 13A) from the syringe 502, a user must first depress the release member 528 towards the fluid chamber 508 in order to disengage the engagement between the front edge 582 of the release member 528 and the locking edge 584 on the guide 580.

Once released, a user may apply a lateral force to the release member 528 in order to push the sliding member 518 forwards. This can be seen in FIG. 13B. FIG. 13B also shows the situation in which a hub 162 had been attached to the syringe 502. As the sliding member 518 is pushed forwards, it advances the hub 162 along the fluid transfer tip 504 (not visible in FIG. 13B) and thereby releases the friction fit. The movement also results in movement of the second segment 516 relative to the first segment 514 such that the separable collar 512 is moved to an open configuration thereby allowing disengagement between the threaded portion 520 on the second segment 516 and the flange 166, i.e. the corresponding engagement portion, on the hub 162. In this particular embodiment, it is the threaded portion 520 which acts on the flange 166 of the hub 162 during the lateral movement of the second segment 516 in order to advance the hub 162 along the fluid transfer tip 504. The threaded portion 520 therefore also acts as a disconnection portion.

Once the friction fit between the hub 162 and the fluid transfer tip 504 has been overcome and the second segment 516 has been advanced sufficiently such that it provides an open configuration allowing the engagement to be released, the hub 162 may freely fall away, for example under gravity. This is depicted in FIG. 13C. Of course, whether the hub 162 is free to fall away from the syringe 502, specifically away from the second segment 516 will depend on the orientation of the syringe 502.

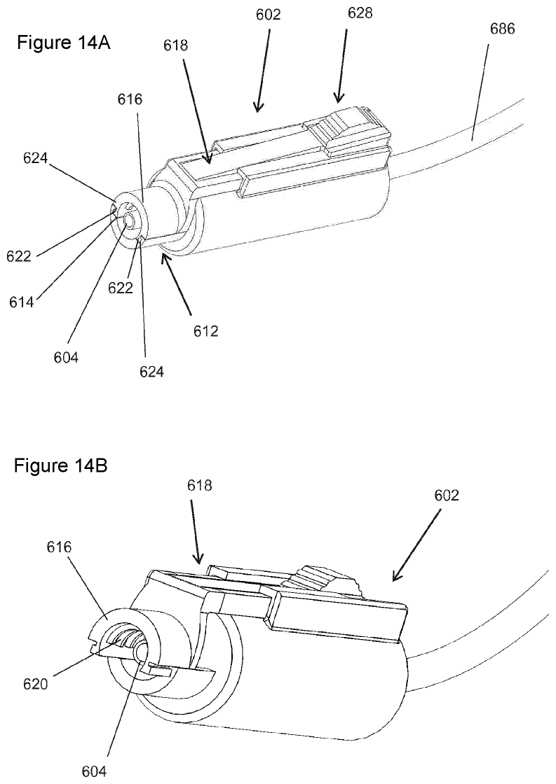

FIG. 14A shows a medical device connector in the form of a fluid transfer connector 602 with a fluid hose 686 extending therefrom. The fluid transfer connector 602 is essentially the same as the syringe 502 seen in in FIG. 13A, except that instead of a fluid chamber 508 for storing fluid, the fluid transfer connector 602 comprises a fluid passage (not shown) extending through the fluid transfer connector 602 from the hose 686 to the fluid transfer tip 604. The fluid transfer connector 602 comprises a sliding member 618 and release member 628 that function in the same manner as the sliding member 518 and release member 528 seen in FIG. 13A. The fluid transfer connector 602 comprises a separable collar 612 that is largely the same as the separable collar 512 of the embodiment seen in in FIG. 13A, except that each of the first segment 614 and second segment 616 each comprises a tongue 622 and groove 624 for engaging with a correspond tongue 622 or groove 624 on the other of the first segment 614 or second segment 616. As will be appreciated by those skilled in the art, the tongues 622 and grooves 624 act to prevent the second segment 616 from moving radially away from the first segment 614.

FIG. 14b shows a perspective view of the fluid transfer connector 602 with the sliding member 618 advanced forward such that the second segment 616 is advanced forward with respect to the fluid transfer tip 604. It can be seen in FIG. 14b that the second segment 616 comprises a threaded portion 620 on its surface arranged concentrically with the fluid transfer tip 604.

In the embodiments seen in FIGS. 13A-13C and 14A-14B, the sliding disconnection member 518, 618 of each embodiment may be provided with a resilient bias which tends to push the sliding disconnection member 518, 618 towards its first position correspond to the separable collar 512, 612 being in a closed configuration. The resilient bias may be provided by any suitable means for example a resilient bias provided by a separate spring member, or an integral resilient bias provided by the sliding disconnection member 518, 618 itself.

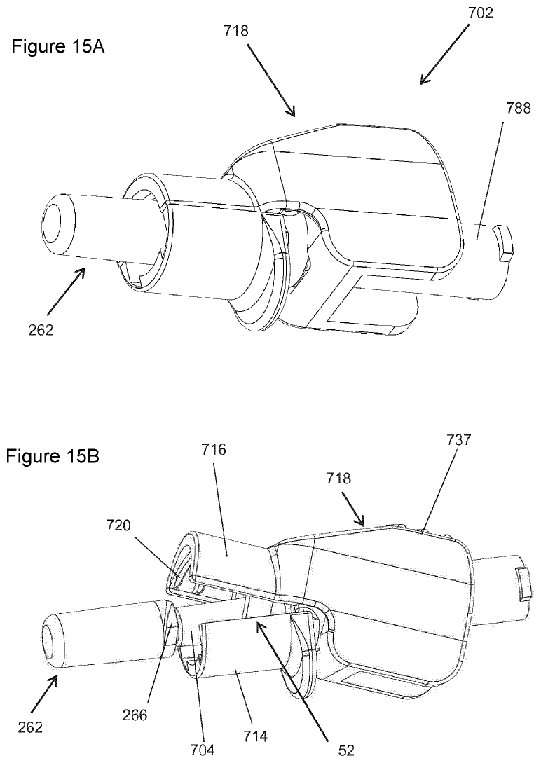

FIG. 15A shows a perspective view of another medical device connector in the form of a fluid transfer connector 702. Attached to the fluid transfer connector 702 is a Luer hub 262. The fluid transfer connector 702 comprises a fluid connection port 788 to which another device or hose may be attached. The lever member 718 whilst shaped slightly differently to previous embodiments functions in essentially the same way. Unlike previous embodiments, the fluid transfer connector 702 does not comprise a release member. Instead, the lever member 718 may be held in the position seen in FIG. 15A, for example, by a resilient bias. FIG. 15B shows the fluid transfer connector 702 following operation of the lever member 718. Here it can be seen that the lever member 718 has moved the second segment 716 away from both the fluid transfer tip 704 and the first, fixed, segment 714 into an open configuration, thereby releasing the engagement between the threaded portion 720 and the corresponding flange 266 on the Luer hub 262. Further, the Luer hub 262 has been advanced along, and in fact separated from, the fluid transfer tip 704 by the disconnecting portion in the form of a forked section 52. A top surface of the lever member 718 may be provided with gripping features 737 to improve a user's grip when operating the lever member 718.

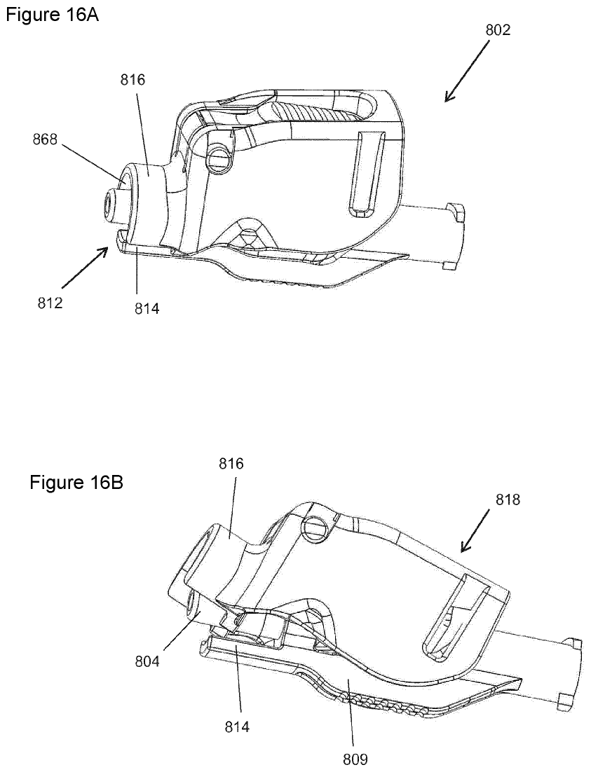

FIG. 16A shows a perspective view of a further embodiment of a medical device connector in the form of a fluid transfer connector 802 (rather than a syringe). The fluid transfer connector 802 is largely the same as the fluid transfer connector 702 seen in FIG. 15A, however the separable collar 812 has a different form. Unlike previous embodiments wherein the separable collar has a 50:50 split with regard to the extent to which each segment extends around the collar, it can be seen in FIG. 16A that the first segment 814 extends around significantly less of the collar than the second segment 816. The second segment 816 is shaped such that at least part of its internal surface defines a space that prevents misconnection, i.e. it defines a specific space suitable for allow a specific type of hub to be attached to the fluid transfer connector 802. It is not essential that the entire space 868 is designed to accommodate a specific type of hub so long as at least a portion of the space 868 is designed to accommodate the specific type of hub. Additionally, in this specific embodiment, neither the first segment nor the second segment 816 comprises any engagement features on their surfaces facing the fluid transfer tip 804. Accordingly, the fluid transfer connector 802 may be used for connection to a hub which does not require additional engagement, e.g. a Luer-Slip hub.

FIG. 16B shows the fluid transfer connector 802, seen in FIG. 16A, following operation of the lever member 818. As the second segment 816 is moved away from the first segment 814 into the open configuration, it is apparent that the first segment 814 extends directly from a main body 809 of the fluid transfer connector 802 and is in a fixed position relative to the fluid transfer tip 804.

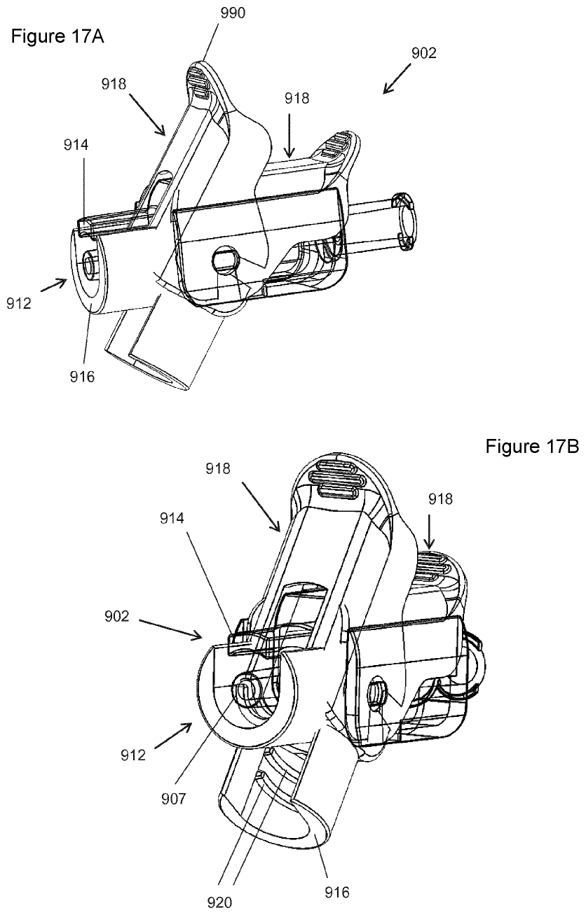

FIGS. 17A and 17B show two different perspective views of another embodiment of a medical device connector in the form of a fluid transfer connector 902. The fluid transfer connector 902 comprises a separable collar 912 having a similar split between the first segment 914 and second segment 916 as seen in the embodiment of FIGS. 16A and 16B. The fluid transfer connector 902 comprises an alternative disconnection member in the form of a lever member 918. Instead of applying a downward force to the lever member 918, it is instead operated by a user applying an upward force at the rear portion 990 of the lever member 918. This may be achieved, for example, by a user pushing the rear portion 990 upwards with a thumb or another finger. In each of FIGS. 17A and 17B the lever member 918 is shown in two positions, a first position corresponding to a closed configuration wherein the separable collar 912 is closed, and an open configuration wherein the separable collar 912 is open. In the open configuration seen in FIG. 17B, there is visible the threaded portions 920 that act as engagement features for a hub (not shown) connected to the fluid transfer tip 907.

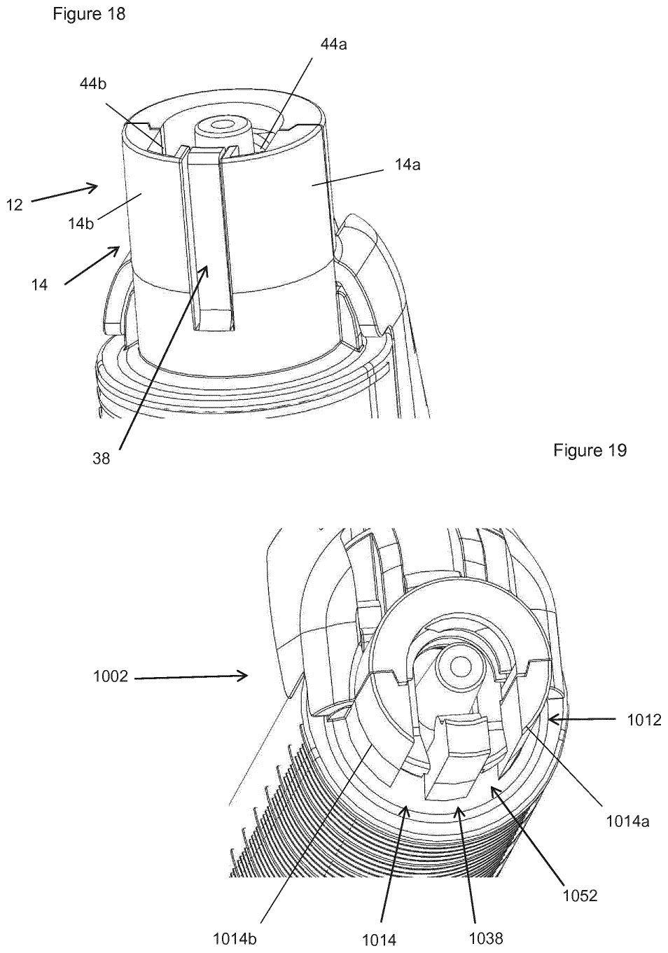

FIG. 18 shows a close-up view focusing on the collar 12 of the first embodiment discussed above. Here it can be seen that the first segment 14, which is fixed relative to the fluid transfer tip 4 is split into a first portion 14a and second portion 14b which are separated by the deceleration feature 38. As mentioned previously, the first portion 14a comprises a slot 44a and the second portion 14b comprises a slot 44b for receiving the forked section 52 of the disconnection portion seen in earlier Figures. Providing the slots 44a, 44b results in a collar 12 which more substantially surrounds the fluid transfer tip 4. However, this is not essential. FIG. 19 shows an alternative embodiment of a medical device connector in the form of a syringe 1002, wherein the separable collar 1012 comprises a first segment 1014 which is separated into a first portion 1014a and second portion 1014b, which are divided by a deceleration feature 1038, wherein instead of slots provided in each of the first and second portions 1014a, 1014b, the first portion 1014a and second portion 1014b are simply arranged such that they are separated from the deceleration feature 1038 to allow the forked section 1052 to pass through.

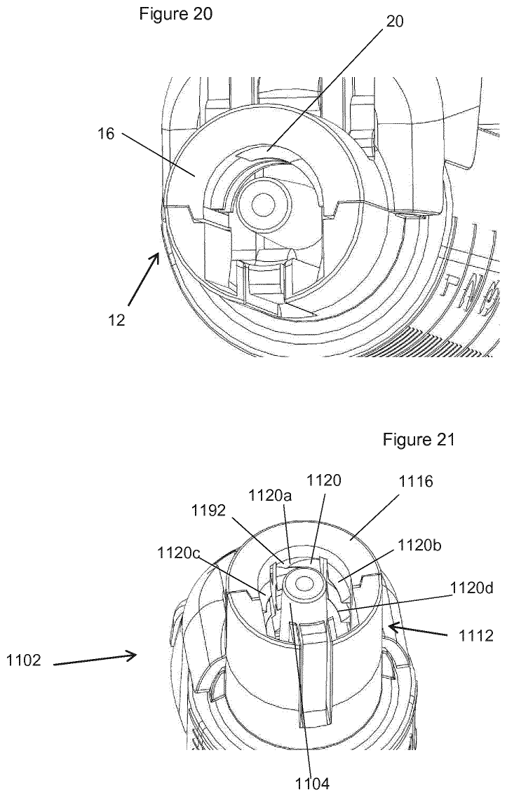

FIG. 20 shows a close-up view of the collar 12 of the syringe 2 seen in FIG. 1. As can be seen in this close-up view, the threaded portion 20 extends continuously along the inside surface of the second segment 16. FIG. 21 shows an alternative embodiment of a syringe 1102 and focusses on the separable collar 1112. The second segment 1116 is provided with a threaded portion 1120, however the threaded portion 1120 is discontinuous and is split into discrete portions 1120a, 1120b, 1120c, 1120d. Each of these discrete portions 1120a, 1120b, 1120c, 1120d are separated from one another, yet still arranged following a helical threaded path such that they effectively provide a thread. The discrete sections 1120a, 1120b, 1120c, 1120d each individually serve to latch onto a hub attached to the syringe 1102. In this particular embodiment each discrete section 1120a, 1120b, 1120c, 1120d is provided with a chamfered edge 1192. This may allow a hub to be pushed onto the fluid transfer tip 1104 and slip past each of the discrete sections 1120a, 1120b, 1120c, 1120d and thus become engaged.

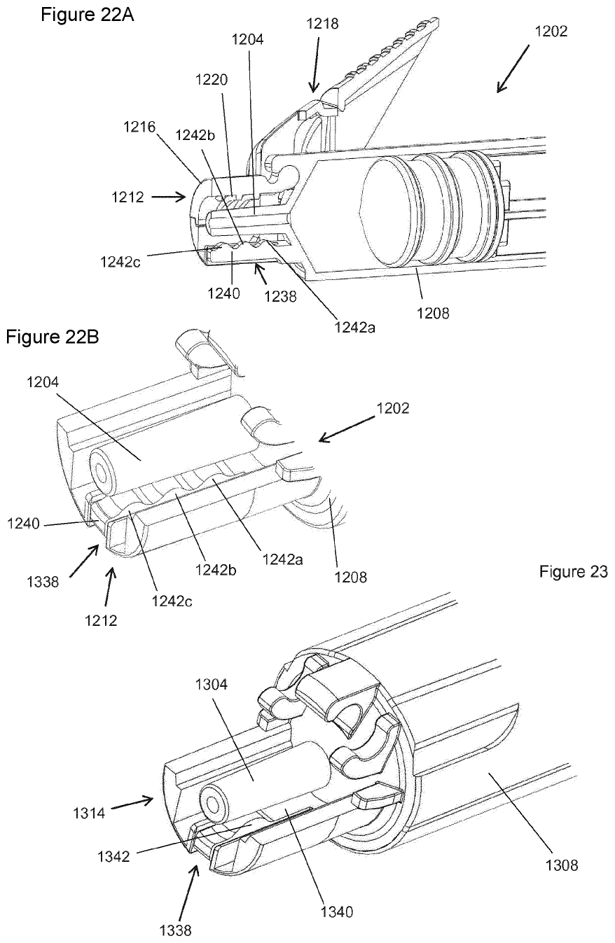

FIG. 22A shows a cross-sectional view of a medical device connector in the form of a syringe 1202 focusing on the deceleration feature 1238 and FIG. 22B shows a perspective view of the syringe 1202 focusing on the separable collar 1212 end of the syringe 1202. As can be seen in each of these Figures, the deceleration feature 1238 is provided by an arm 1240 which extends from a front end of the fluid chamber 1208. The arm 1240 comprises three protrusions 1242a, 1242b, 1242c. As can be seen most clearly in FIG. 22A, the three protrusions extend towards the fluid transfer tip 1204. A hub which is attached to the syringe 1202 will be attached to the fluid transfer tip 1204 by a friction fitting, and engaged by the engagement feature provided by a thread 1220 on the second segment 1216. When a user disconnects the hub by operating the lever member 1218, the hub will advance along the fluid transfer tip 1204. As the hub advances, its movement will be resisted, and slowed, by interaction between a part of the hub, for example a flange thereon, and the deceleration feature 1238, specifically the protrusions 1242a, 1242b, 1242c. In this particular embodiment, the hub must pass over each of the three protrusions 1242a, 1242b, 1242c in order to become fully separated from the syringe 1202. As the hub approaches the last protrusion 1242c, the friction fit between the hub and the fluid transfer tip 1204 may be largely, if not completely overcome, and thus a user may release the hub in a more controlled manner. The deceleration feature 1238 helps to prevent the hub from being dangerously, and forcibly, ejected from the fluid transfer tip 1204, by slowing down its motion as the friction fit is overcome. As will be appreciated by those skilled in the art, typically in order to separate a hub from a friction fit a threshold force must be provided. As the force being applied to the hub is increased and the threshold force is reached, it is known that hubs are often caused to become dangerously ejected from the device they are attached to. Accordingly, by providing resistance to the hubs motion via the deceleration feature 1238, this dangerous ejection can be avoided.

FIG. 23 shows a perspective view of a fluid chamber 1308 of a further embodiment. A deceleration feature 1338 is provided by an arm 1340 extending from the forward end of the fluid chamber 1308. The deceleration feature 1338 comprises a single protrusion 1242 extending from the arm 1340 towards the fluid transfer tip 1304. The deceleration feature 1338 functions in a similar manner to that described above with respect to FIGS. 22A and 22B, except that the advancement of the hub along the fluid transfer tip 1304 is only resisted by a single protrusion 1342, rather than three. Accordingly, once the hub has passed the single protrusion 1342, the deceleration feature 1338 will no longer decelerate advancement of the hub.

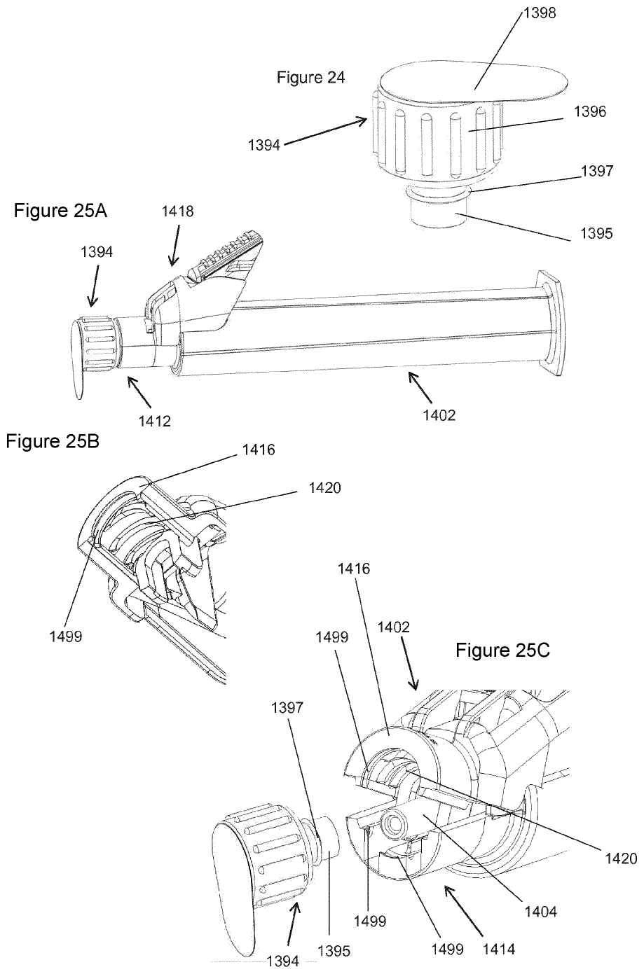

FIG. 24 shows a perspective view of a disinfecting cap 1394. The disinfecting cap 1394 defines a chamber 1396 containing disinfecting liquid such as an alcohol and/or any disinfecting solution with antiseptic, antimicrobial, antibacterial and/or anticoagulant properties. The disinfecting liquid may be retained by an absorbent material (not visible in this Figure) such as a sponge inside the chamber 1396. The disinfecting cap 1394 has an interior surface with a set of threads (not visible in this Figure) for mating with a set of threads on a hub when the disinfecting cap 1394 is connected in use. The disinfecting cap 1394 is sealed by a removable lid 1398 (e.g. foil, paper or plastic material) that can be peeled away when the disinfecting cap 1394 is ready to be used. When the disinfecting cap 1394 is connected to the port hub of a catheter or related tubing, the catheter is sealed to block the ingress of pathogens and contaminants and instead the hub is disinfected by the disinfecting liquid. Unlike conventional disinfecting caps, such as the Curos cap by 3M, the disinfecting cap 1394 further comprises a connection portion 1395 which may be used to grip the disinfecting cap 1394 in order to manipulate the disinfecting cap 1394, e.g. when applying it to a hub, but also may be used as a means for storing the disinfecting cap 1394. In this particular embodiment, the connection portion 1395 is in the form of a hub with a tapered internal surface allowing it to be connected to a further device, e.g. a fluid transfer tip. The connection portion 1395 further comprises a circumferential flange 1397 which may allow the connection portion 1395 to be further engaged by a device. In addition, or alternatively to the flange 1397, an external thread or another type of external engagement feature which can be engaged by a suitable device may be provided on the connection portion 1395.

FIG. 25A shows a further embodiment of a medical device connector in the form of a syringe 1402 which can be pre-filled with a flush solution e.g. for use with an indwelling, central venous catheter. The syringe 1402 is similar to the syringe 2 seen in FIG. 1, except that it is pre-filled with a flush solution. In use, the flush solution is injected into a hub port of the catheter to clean and disinfect the catheter. In this embodiment the pre-filled syringe 1402 is sealed by a disinfecting cap 1394 connected to the fluid transfer tip (not visible in this Figure). The disinfecting cap 1394 is attached to the fluid transfer tip of the syringe 1402 and the flange 1397, as seen in FIG. 24, is engaged by a dedicated groove (not visible in FIG. 25A) on the separable collar 1412.

FIG. 25B shows an underside view focusing on the second segment 1416 of the separable collar 1412. As can be seen in this view, in addition to a threaded portion 1420 for engaging with a hub, in use, the second segment 1416 further comprises a groove 1499 arranged at a front end of the second segment 1416. This groove 1499 is arranged, to receive the flange 1397 of the disinfecting cap 1394 when the disinfecting cap 1394 is connected to the fluid transfer tip 1404 of the syringe 1402 (as seen in FIG. 25C).

FIG. 25C shows the front end of the syringe 1402 following disconnection of the disinfecting cap 1394. As can be seen in this Figure, the groove 1499 provided on the second segment 1416 also extends onto the first segment 1414 such that the first segment 1414, also receives and engages the flange 1397. Disconnection of the disinfecting cap 1394 occurs in an identical manner to disconnection of a hub as described previously. The disinfecting cap 1394 can be disconnected immediately before use of the flush solution, and thereafter easily mounted onto the port hub.

In other embodiments, the pre-filled syringe 1402 may of course be sealed by a regular cap connected to the tip. In any of these embodiments, one-handed operation of the lever member is advantageous for convenient cap removal prior to a flushing procedure.

With reference to FIGS. 25A-25C, the disinfecting cap 1394 may be attached to the syringe 1404 with the separable collar 1412 in the closed configuration. The flange 1397 provided on the disinfecting cap 1394, and the groove 1499 provided on the separable collar 1412, may both be designed such that at least one of the flange 1397 or groove 1499 at least partially deforms or flexes, due to inherent flexibility within the material from which they are made, to allow the disinfecting cap 1394 to be attached to the syringe 1404. The disinfecting cap 1394 may therefore be ‘clicked’ into place. The flange 1397 and/or groove 1499 may be designed such that the disinfecting cap 1394 may be pushed onto the syringe 1402 but only be disconnected through use of the lever member 1418. The Applicant has recognized that such a device is particularly advantageous as it may promote aseptic working procedures by helping to avoid a user, e.g. a medical practitioner, from coming into contact with the fluid transfer tip 1404.

Additionally, as can be seen in FIG. 25C, the groove 1499 is located directly at the opening to the collar 1412 at the forward end of the first segment 1414 and second segment 1416. The Applicant has appreciated that arranging the groove 1499 in this forward position is advantageous as it means that, at least in embodiments wherein the disinfecting cap 1394 is devoid of any other engaging means on the connecting portion 1395 thereof, the disinfecting cap 1394 does not interfere with the threaded portion 1420 on the second segment. As the disinfecting cap 1394 may be stored on the end of the syringe 1402 for a significant period of time, for example up to 5 years, avoiding contact with between the disinfecting cap 1394 and the threaded portion 1420 ensures that the threaded portion 1420 does not become deformed or fatigued prior to its eventual use in engaging with a hub and thus ensures that it will be fit for purpose.

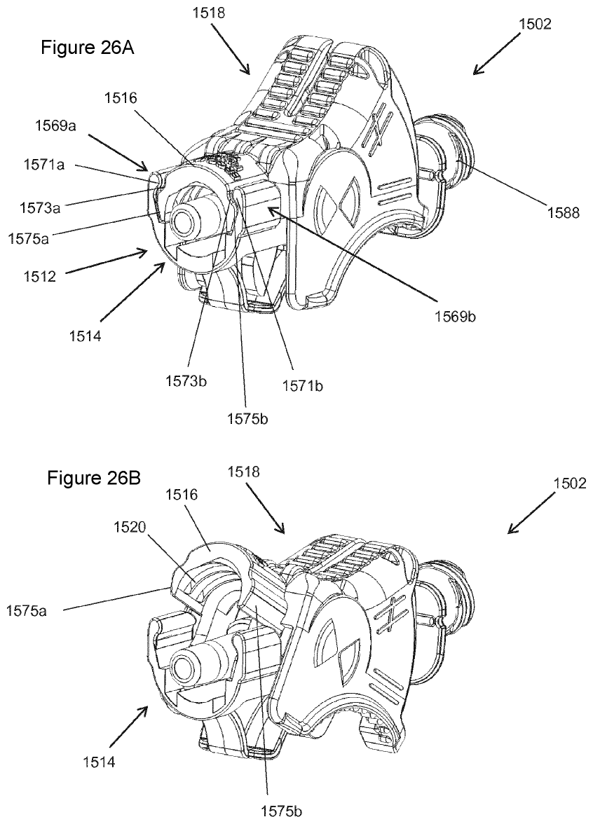

FIG. 26A shows a perspective view of another medical device connector embodying the invention in the form of a fluid transfer connector 1502. Similarly to earlier embodiments, the fluid transfer connector 1502 comprises a fluid connection port 1588 to which another device or hose may be attached. The fluid transfer connector 1502 comprises a disconnection member in the form of a lever member 1518 which, whilst shaped slightly differently to previous embodiments, functions in essentially the same way. Similarly to previous embodiments, the fluid transfer connector comprises a separable collar 1512 which comprises a first arcuate segment 1514 and second arcuate segment 1516. The second arcuate segment 1516 extends from the lever member 1518.

This particular embodiment seen in FIG. 26A comprises a first latching feature 1569a and second latching feature 1569b arranged to lock the first arcuate segment 1514 and second arcuate segment 1516 together in the closed configuration depicted in FIG. 26A. The latching feature 1569a comprises a first protrusion 1571a and second protrusion 1571b at opposite extremes of the first arcuate segment 1514. The first protrusion 1571a and second protrusion 1571b engage with a first recess 1573a and second recess 1573b on the second arcuate segment 1516 to lock the first and second arcuate segments 1514, 1516 together to form a closed collar which is positively latched, i.e. locked, in position. As discussed previously, latching the first and second arcuate segments 1514, 1516 together may help to prevent a user from inadvertently separating the first and second arcuate segments 1514, 1516, for example when trying to attach a non-compatible hub. This may therefore further help to prevent the connection of non-compatible hubs.

As can also be seen in FIG. 26A, the second arcuate segment 1516 further comprises a first chamfered edge 1575a and second chamfered edge 1575b, the purpose of which will be described in more detail below.

In order to separate the second arcuate segment 1516 from the first arcuate segment 1514, the first latching feature 1569a and second latching feature 1569b must first be disengaged. In this particular embodiment, the first arcuate segment 1514 and second arcuate segment 1516 are slight deformable, at least in the portion proximal to the first and second protrusions 1571a, 1571b and first and second recesses 1573a, 1573b. As will be appreciated, when a user applies a force to the lever member, e.g. by depressing the lever member 1518, the lever member 1518 will transfer and amplify the applied force which will initially go towards deforming the first and second arcuate segment 1514, 1516 such that the first and second latching features 1569a, 1569b are disengaged. Continued application of a force by the user to the lever 1518 will then cause the lever member 1518 to pivot thereby moving the second arcuate segment 1516 away from the first arcuate segment 1514.

FIG. 26B shows a perspective view of the fluid transfer connector 1502, seen in FIG. 26A, following operation of the lever member 1518, i.e. after the first and second latching features 1569a, 1569b have been disengaged and after it has been pivoted. As seen in this Figure, once the lever member 1518 has been operated, the second segment 1516 is separated from the first segment 1514 into an open configuration. Release of a hub attached to the fluid transfer connector 1502 is achieved in the same manner as with previous embodiments, for example as described above with reference to FIGS. 15A and 15B. It can be seen in FIG. 26B that the second segment 1516 comprises a threaded portion 1520 as an engagement feature on its inner cylindrical surface.

With reference to both FIGS. 26A and 26B, with the fluid transfer connector 1502 in the configuration seen in FIG. 26B, when a user wishes for the collar 1512 to be in the closed configuration, for example to allow them to attach a hub to the fluid transfer connector 1502, the lever member may be moved back to the position seen in FIG. 26A. The chamfered edges 1575a, 1575b provided on the second arcuate segment 1516 allow the second arcuate segment 1516 to move past the first and second protrusions 1571a, 1571b on the first arcuate segment 1514. As will be appreciated, as the chamfered edges 1575a, 1575b move past the first and second protrusions 1571a, 1571b, slight deformation of at least one of the first or second arcuate segments 1515, 1516 may occur to permit this movement and thus allow it to move into the closed, and locked, position seen in FIG. 26A. The provision of these chamfered edges 1575a, 1575 may allow the lever member 1518 to move into the position seen in FIG. 26A under a resilient bias, if provided, without requiring further interaction from a user.

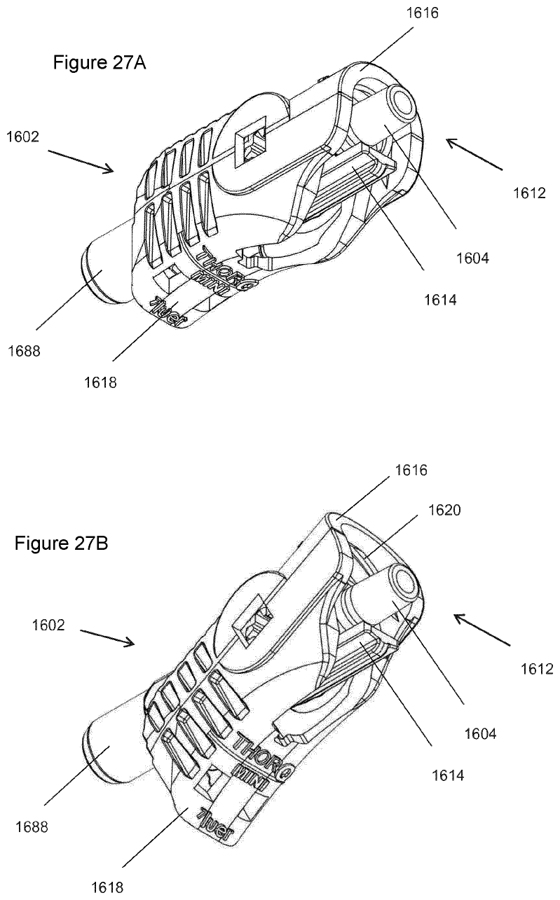

FIG. 27A shows a perspective view of another embodiment of a medical device connector in the form of a fluid transfer connector 1602. Similarly to earlier embodiments, the fluid transfer connector 1602 comprises a fluid connection port 1688 to which another device or hose may be attached. The fluid transfer connector 1602 comprises a disconnection member in the form of a lever member 1618 which, whilst shaped slightly differently to previous embodiments, functions in essentially the same way. Similarly to previous embodiments, the fluid transfer connector 1602 comprises a separable collar 1612 which comprises a first arcuate segment 1614 and second arcuate segment 1616. The second arcuate segment 1616 extends from the lever member 1618. It can be seen that the angular extent of the first arcuate segment 1614 is much less than the angular extent of the second arcuate segment 1616. The first and second arcuate segments 1614, 1616 are still arranged concentrically relative to the fluid transfer tip 1604, but in this embodiment the second arcuate segment 1616 has an outer profile that is generally U-shaped. The first arcuate segment 1614 sits inside the U-shaped second segment 1616 in the closed configuration seen in FIG. 27A.

FIG. 27B shows a perspective view of the fluid transfer connector 1602, seen in FIG. 27A, following operation of the lever member 1618. As seen in this Figure, once the lever member 1618 has been operated, the second segment 1616 is separated from the first segment 1614 into an open configuration of the collar 1612. Release of a hub connected to the fluid transfer tip 1604 of the connector 1602 is achieved in the same manner as with previous embodiments, for example as described above with reference to FIGS. 15A and 15B. It can be seen in FIG. 27B that the second segment 1616 comprises a threaded portion 1620 as an engagement feature on its inner cylindrical surface.

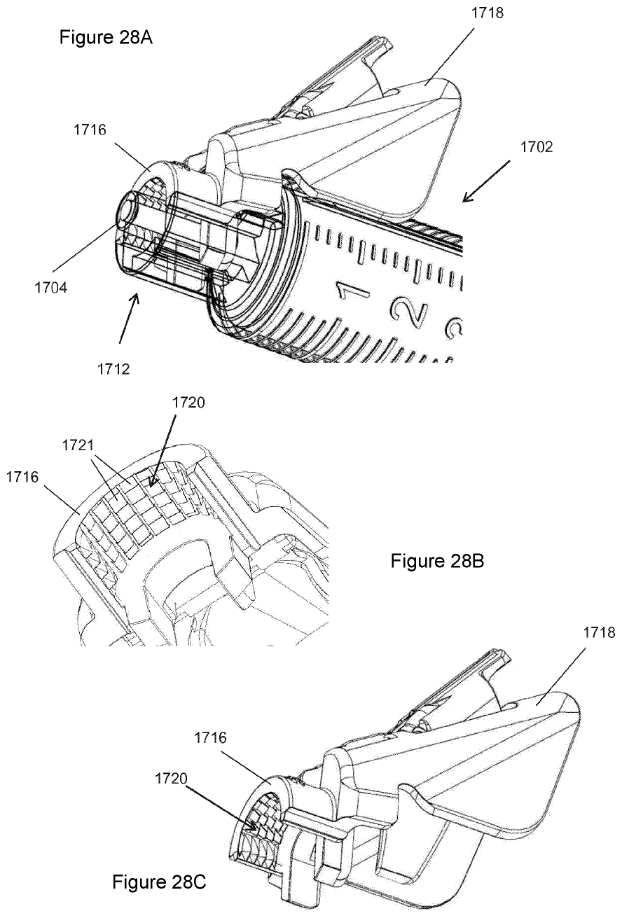

FIGS. 28A-28C show a further embodiment of a medical device connector in the form of a syringe 1702. The syringe 1702 includes a fluid transfer tip 704, a separable collar 1712 extending around the fluid transfer tip 704, and a disconnection member 1718 in the form of a lever member. The lever member 1718 pivots in order to open the collar 1712. One of the main differences between this syringe 1702 and the syringe 2 already described in relation to FIGS. 1-6 is the way in which the second segment 1716 engages with a hub (not shown) connected to the fluid transfer tip 1704 in use. As is most clearly seen in FIGS. 28B and 28C, the second segment 1716 comprises multiple discrete engagement features 1721 in a helical arrangement 1720 extending around the fluid transfer tip 1704. Each engagement feature 1721 is a latch member in the shape of an angled tooth. Such latch members 1721 may be arranged helically, as shown here, so as to function like a threaded engagement portion. A user may need to screw on a hub when the collar 1712 is in its closed configuration. Alternatively, although not illustrated, such latch members 1721 may instead be arranged concentrically around the tip 1704 without following a helical path. A user may then need to open the collar 1712 in order to connect a hub.

As will be appreciated by those skilled in the art, any of the embodiments described above may be modified in an appropriate manner such that they are suitable for connection with a specific type of hub, e.g. a Luer Fit, NRFit or ENFit hub. For example, this may involve providing a specific fluid transfer tip and/or collar.

While the invention has been described with reference to exemplary embodiments and applications scenarios, it will be understood by those skilled in the art that various changes may be made and equivalents may be substituted for elements thereof without departing from the scope of the claims. Therefore, it is intended that the invention not be limited to the particular embodiments disclosed, but that the invention will include all embodiments falling within the scope of the appended claims and can be applied to various application in the industrial as well as commercial field.

What is claimed is:

1. A medical device connector for connecting, in use, to a hub, the medical device connector comprising:
a fluid transfer tip comprising a tapered surface for creating a friction fitting between the fluid transfer tip and a hub connected to the fluid transfer tip in use;
a separable collar, comprising at least a first arcuate segment and a second arcuate segment, which extend at least partially around the fluid transfer tip, wherein the first segment is arranged in a fixed position relative to the fluid transfer tip and the second segment is moveably mounted relative to the fluid transfer tip and the first segment;
wherein the second segment comprises one or more engagement features for engaging with at least one corresponding engagement feature of a hub connected to the fluid transfer tip in use;
the separable collar having a closed configuration in which the second segment is positioned relative to the fluid transfer tip so as to extend around a hub connected to the tip in use and to engage with the hub by engagement between the one or more engagement features of the second segment and the at least one corresponding engagement feature of the hub, and an open configuration in which the second segment is moved relative to the fluid transfer tip and the first segment, so as to allow disengagement of the one or more engagement features from the at least one corresponding engagement feature; and
a moveable disconnection member arranged such that movement of the disconnection member moves the second segment of the separable collar from a first position, corresponding to the closed configuration of the separable collar, to a second position, corresponding to the open configuration of the separable collar, and wherein movement of the disconnection member also releases the friction fitting between the fluid transfer tip and the hub by advancing the hub along the tapered surface of the fluid transfer tip.

2. The connector of claim 1, wherein the one or more engagement features are in a helical arrangement extending around the fluid transfer tip.

3. The connector of claim 1, wherein the one or more engagement features comprise at least one threaded portion for engaging with a corresponding threaded portion or flange of a hub connected to the fluid transfer tip in use.

4. The connector of claim 1, wherein the one or more engagement features comprises at least one latch member.

5. The connector of claim 1, wherein the fluid transfer tip extends along an axis that is surrounded by the separable collar, and the fluid transfer tip extends no further than an outer surface of the separable collar.

6. The connector of claim 1, wherein the fluid transfer tip extends along an axis that is surrounded by the separable collar, and the separable collar extends substantially all the way around the axis of the fluid transfer tip in the closed configuration.

7. The connector of claim 1, wherein the first and second arcuate segments mate together in the closed configuration so as to form a closed collar extending substantially all the way around the fluid transfer tip.

8. The connector of claim 1, wherein the first and second arcuate segments are arranged concentrically relative to the fluid transfer tip.

9. The connector of claim 1, wherein at least one of the first and second arcuate segments has an outer profile that is generally U-shaped.

10. The connector of claim 1, wherein at least the first arcuate segment is arranged in a fixed position having a predefined spacing from the tapered surface of the fluid transfer tip.

11. The connector of claim 10, wherein the predefined spacing is compliant with ISO 80369-3, ISO 80369-6 or ISO 80369-7.

12. The connector of claim 1, wherein the separable collar comprises a deceleration feature arranged to interact with a hub connected to the fluid transfer tip in use so as to at least partially resist advancement of the hub along the fluid transfer tip, and preferably the first segment comprises the deceleration feature

13. A system comprising a hub and medical device connector;
wherein the hub comprises:
a connection feature arranged at a first end of the hub for connecting the hub to the medical device connector; and
wherein the medical device connector comprises:
a fluid transfer tip comprising a tapered surface for creating a friction fitting between the fluid transfer tip and the connection feature on the hub when the hub is connected to the medical device connector;
a collar arranged to extend at least partially around the fluid transfer tip, and around at least part of the hub when the hub is connected to the fluid transfer tip, and wherein the collar comprises a deceleration feature; and
a disconnection member arranged such that when the hub is connected to the fluid transfer tip, movement of the disconnection member from a first position to a second position advances the hub along the fluid transfer tip so as to release the friction fitting between the fluid transfer tip and the hub and wherein the advancement of the hub along the fluid transfer tip is at least partially resisted by an interaction between the deceleration feature and the hub.

14-15. (canceled)

16. The system of claim 13, wherein the deceleration feature is at least partially deformable and/or displaceable such that as the hub is advanced across the deceleration feature, the deceleration feature deforms and/or displaces to allow the hub to advance along the fluid transfer tip.

17-18. (canceled)

19. The connector of claim 1, wherein the disconnection member is locked in the first position until actively released, and further comprising a release member operable to release the disconnection member.

20. (canceled)

21. The connector of claim 19, wherein the release member comprises deformable side legs which extend towards and rest on an outer surface of the body member and arranged such that, as the release member is pressed down towards the body member, the side legs are forced to splay outwards around the body member to contact the disconnection member and further operation of the release member thereby drives movement of the disconnection member.

22. (canceled)

23. The connector of claim 1, wherein the disconnection member comprises a disconnection portion arranged to advance along the tapered surface of the tip to advance and push off the hub from the friction fitting.

24. The connector of claim 1, wherein the disconnection member is a pivotally mounted lever member.

25-26. (canceled)

27. A medical device connector for connecting, in use, to a hub, the medical device connector comprising:
a fluid transfer tip comprising a tapered surface for creating a friction fitting between the fluid transfer tip and a hub connected to the fluid transfer tip in use;
a separable collar, comprising at least a first arcuate segment and a second arcuate segment, which extend at least partially around the fluid transfer tip, wherein at least one of the first and second segments is moveably mounted relative to the fluid transfer tip, and wherein each of the first and second segments comprises a surface facing the fluid transfer tip that is devoid of engagement features;
the separable collar having a closed configuration in which the second segment is positioned relative to the fluid transfer tip so as to extend around a hub connected to the tip in use, and an open configuration in which the first and/or second segment is moved relative to the fluid transfer tip; and
a moveable disconnection member arranged such that movement of the disconnection member moves the first and/or second segment of the separable collar from a first position, corresponding to the closed configuration of the separable collar, to a second position, corresponding to the open configuration of the separable collar, and wherein movement of the disconnection member also releases the friction fitting between the fluid transfer tip and the hub by advancing the hub along the tapered surface of the fluid transfer tip.

28. The connector of claim 27, wherein the first segment is arranged in a fixed position relative to the fluid transfer tip and the second segment is moveably mounted relative to the fluid transfer tip and the first segment.

29-32. (canceled)
